(19) United States
(12) Patent Application Publication
(10) Pub. No.: US 2025/0195533 A1
(43) Pub. Date: Jun. 19, 2025

(54) COMBINATION THERAPIES FOR THE TREATMENT OF CANCER

(71) Applicant: Erasca, Inc., San Diego, CA (US)

(72) Inventors: Leenus MARTIN, San Diego, CA (US); Leslie Harris BRAIL, Newton, MA (US); Robert Field SHOEMAKER, San Diego, CA (US)

(21) Appl. No.: 18/256,091

(22) PCT Filed: Dec. 10, 2021
(86) PCT No.: PCT/US2021/062927

§ 371 Date: Jun. 6, 2023

Related U.S. Application Data
(60) Provisional application No. 63/283,035, filed on Nov. 24, 2021.
(60) Provisional application No. 63/277,555, filed on Nov. 9, 2021.
(60) Provisional application No. 63/214,736, filed on Jun. 24, 2021.
(60) Provisional application No. 63/124,669, filed on Dec. 11, 2020.

(51) Int. Cl. A61K 31/5383; A61K 9/00; A61K 31/519; A61P 35/00

(52) U.S. Cl. CPC A61K 31/5383; A61K 9/0053; A61K 31/519; A61P 35/00

(57) ABSTRACT

The present disclosure provides methods of treating cancer with combination therapies of a SHP2 inhibitor and a KRAS G12C inhibitor.

### CROSS-REFERENCE

This application claims the benefit of U.S. Provisional Patent Application No. 63/124,669 filed Dec. 11, 2020; U.S. Provisional Patent Application No. 63/214,736 filed Jun. 24, 2021; U.S. Provisional Patent Application No. 63/277,555 filed Nov. 9, 2021; and U.S. Provisional Patent Application No. 63/283,035 filed Nov. 24, 2021; each of which is incorporated herein by reference in its entirety.

### BACKGROUND OF THE INVENTION

Src Homology-2 phosphatase (SHP2) is a non-receptor protein phosphatase ubiquitously expressed in various tissues and cell types (see reviews: Tajan M et al., Eur J Med Genet 2016 58 (10): 509-25; Grossmann K S et al., Adv Cancer Res 2010 106:53-89). SHP2 is composed of two Src homology 2 (N—SH2 and C—SH2) domains in its NH2-terminus, a catalytic PTP (protein-tyrosine phosphatase) domain, and a C-terminal tail with regulatory properties. At the basal state, the intermolecular interactions between the SH2 domains and the PTP domain prevent the access of substrates to the catalytic pocket, keeping SHP2 into a closed, auto-inhibited conformation. In response to stimulation, SHP2 activating proteins bearing phosphor-tyrosine motifs bind to the SH2 domains, leading to exposure of active site and enzymatic activation of SHP2.

### SUMMARY OF THE INVENTION

The present embodiments disclosed herein generally relate to compositions and methods related to combination therapies to treat cancer utilizing a SHP2 inhibitor in conjunction with a KRAS G12C inhibitor, including while providing an unexpected degree synergy.

SHP2 plays important roles in fundamental cellular functions including proliferation, differentiation, cell cycle maintenance and motility. By dephosphorylating its associated signaling molecules, SHP2 regulates multiple intracellular signaling pathways in response to a wide range of growth factors, cytokines, and hormones. Cell signaling processes in which SHP2 participates include the RAS-MAPK (mitogen-activated protein kinase), the PI3K (phosphoinositol 3-kinase)-AKT, and the JAK-STAT pathways.

SHP2 also plays a signal-enhancing role on this pathway, acting downstream of RTKs and upstream of RAS. One common mechanism of resistance for pharmacological inhibition of MAPK signaling involves activation of RTKs that fuel reactivation of the MAPK signaling. RTK activation recruits SHP2 via direct binding and through adaptor proteins. Those interactions result in the conversion of SHP2 from the closed (inactive) conformation to open (active) conformation. SHP2 is an important facilitator of RAS signaling reactivation that bypasses pharmacological inhibition in both primary and secondary resistance. Inhibition of SHP2 achieves the effect of globally attenuating upstream RTK signaling that often drives oncogenic signaling and adaptive tumor escape (see Prahallad, A. et al. Cell Reports 12, 1978-1985 (2015); Chen Y N, Nature 535, 148-152 (2016)), which is incorporated herein by reference in its entirety for all of its teachings, including without limitation all methods, compounds, compositions, data and the like, for use with any of the embodiments and disclosure herein.

In addition to SHP2, the RAS-MAPK signal transduction pathway includes the Ras family of proteins. The family includes three related GTPases (K-, N- and HRAS) that play a role in signal transduction pathways. KRAS, in particular, is known to have numerous mutations indicating an oncogenic state. KRAS mutants, such as mutations occurring at amino acid residue 12 (i.e., G12X), are commonly known to cause cancer. For example, the G12C mutation occurs in about 13% of NSCLC patients, and 1% to 3% of colorectal cancer and solid tumors.

In a first aspect, the present disclosure provides a method of treating a subject having cancer comprising administering to the subject a therapeutically effective amount of a compound of Formula I or its pharmaceutically acceptable salt:

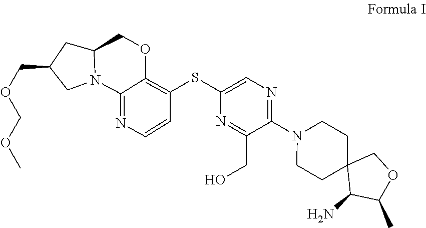 [SMILES: COCOC[C@H]1C[C@H]2COc3c(Sc4cnc(N5CCC6(CC5)CO[C@@H](C)[C@H]6N)c(CO)n4)ccnc3N2C1]

in combination with a KRAS inhibitor.

In some embodiments, the cancer comprises a KRAS G12C mutation.

In some embodiments, the cancer is lung cancer.

In some embodiments, the cancer is non-small cell lung cancer.

In some embodiments, the cancer is esophageal cancer.

In some embodiments, the cancer is pancreatic ductal adenocarcinoma (PDAC).

In some embodiments, the KRAS inhibitor is selected from the group consisting of AMG 510 (sotorasib, LUMAKRAS™), MRTX849 (adagrasib), ARS-3248, GDC-6036, BI 1701963, tipifarnib and BBP-454.

In some embodiments, the KRAS inhibitor is AMG 510.

In some embodiments, the KRAS inhibitor is MRTX849.

In some embodiments, the KRAS inhibitor is ARS-3248.

In some embodiments, the KRAS inhibitor is BI 1701963.

In some embodiments, the method comprises administering a third MAPK pathway inhibitor.

In some embodiments, the administration is oral.

In some embodiments, the dosing of the compound of Formula I is in a range from 20 mg to 400 mg daily.

In some embodiments, the dosing of the KRAS inhibitor is in a range from 1 mg to 1,000 mg daily.

In a second aspect, the present disclosure provides a method of treating a lung or esophageal cancer in a subject comprising orally administering to the subject a therapeutically effective amount of a compound of Formula I or its pharmaceutically acceptable salt:

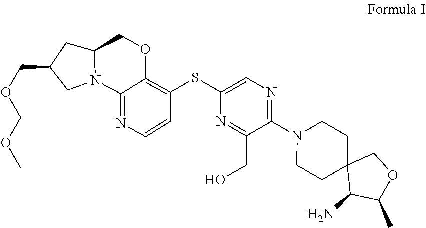 [SMILES: COCOC[C@H]1C[C@H]2COc3c(Sc4cnc(N5CCC6(CC5)CO[C@@H](C)[C@H]6N)c(CO)n4)ccnc3N2C1]

in combination with AMG 510.

In some embodiments, the compound of Formula I is administered once or twice daily.

In some embodiments, AMG 510 is administered once or twice daily.

In some embodiments, the subject is a human.

In a third aspect, the present disclosure provides a method of treating cancer in a subject comprising orally administering to the subject a therapeutically effective amount of a compound of Formula I or its pharmaceutically acceptable salt:

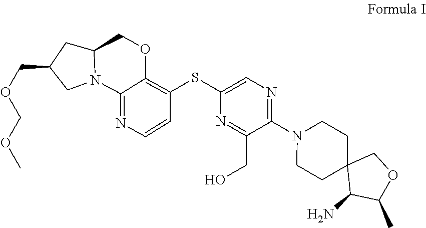 [SMILES: COCOC[C@H]1C[C@H]2COc3c(Sc4cnc(N5CCC6(CC5)CO[C@@H](C)[C@H]6N)c(CO)n4)ccnc3N2C1]

in combination with MRTX849.

In some embodiments, the cancer is lung, colorectal, esophageal or breast cancer.

In some embodiments, the cancer is pancreatic ductal adenocarcinoma (PDAC).

In some embodiments, the compound of Formula I is administered once or twice daily.

In some embodiments, MRTX849 is administered once or twice daily.

In some embodiments, the subject is a human.

In some embodiments, there are provided kits comprising a compound of Formula I or a pharmaceutically acceptable salt thereof and a KRAS inhibitor.

In some embodiments, the compound of Formula I and the KRAS inhibitor are in separate packages.

In some embodiments, the kit further comprises instructions to administer the contents of the kit to a subject for the treatment of cancer.

In some embodiments, the KRAS inhibitor is one or more of AMG 510, MRTX849, ARS-3248, GDC-6036, BI 1701963, tipifarnib and BBP-454.

In some embodiments, the compound of Formula I, or a pharmaceutically acceptable salt thereof, is formulated as a pharmaceutical composition. In some embodiments, the compound of Formula I, or a pharmaceutically acceptable salt thereof, is formulated as an oral composition.

In some embodiments, the compound of Formula I, or a pharmaceutically acceptable salt thereof, is administered once or twice a day. In some embodiments, the compound of Formula I, or a pharmaceutically acceptable salt thereof, is administered once a day. In some embodiments, the compound of Formula I, or a pharmaceutically acceptable salt thereof, is administered twice a day. In some embodiments, the compound of Formula I, or a pharmaceutically acceptable salt thereof, is administered over a continuous 28-day cycle.

In some embodiments, the compound of Formula I, or a pharmaceutically acceptable salt thereof, is administered once a day in the amount of about 10 mg to about 140 mg.

In some embodiments, the compound of Formula I, or a pharmaceutically acceptable salt thereof, is administered once a day for a 3-week cycle, comprising 2 weeks of administration of the compound followed by 1 week of no administration of the compound.

In some embodiments, the compound of Formula I, or a pharmaceutically acceptable salt thereof, is administered once a day for a 4-week cycle, comprising 3 weeks of administration of the compound followed by 1 week of no administration of the compound.

In some embodiments, the compound of Formula I, or a pharmaceutically acceptable salt thereof, is administered over a period of 6 weeks. In some embodiments, the compound of Formula I, or a pharmaceutically acceptable salt thereof, is administered over a period of 8 weeks.

In some embodiments, the compound of Formula I, or a pharmaceutically acceptable salt thereof, is administered 3 times a week. In some embodiments, the compound of Formula I, or a pharmaceutically acceptable salt thereof, is administered on day 1, day 3, and day 5 of the week.

In some embodiments, the compound of Formula I, or a pharmaceutically acceptable salt thereof, is administered 4 times a week.

In some embodiments, the compound of Formula I, or a pharmaceutically acceptable salt thereof, is administered for a 3-week cycle, comprising 2 weeks of administration of the compound followed by 1 week of no administration of the compound.

In some embodiments, the compound of Formula I, or a pharmaceutically acceptable salt thereof, is administered for a 4-week cycle, comprising 3 weeks of administration of the compound followed by 1 week of no administration of the compound.

In some embodiments, the compound of Formula I, or a pharmaceutically acceptable salt thereof, is administered twice a day, two days per week. In some embodiments, the compound of Formula I, or a pharmaceutically acceptable salt thereof, is administered over a period of 8 weeks. In some embodiments, the compound of Formula I, or a pharmaceutically acceptable salt thereof, is administered on day 1 and day 2 of each week.

In some embodiments, the cancer is selected from lung cancer, stomach cancer, liver cancer, colon cancer, kidney cancer, breast cancer, pancreatic cancer, pancreatic ductal adenocarcinoma (PDAC), juvenile myelomonocytic leukemia, neuroblastoma, melanoma, and acute myeloid leukemia.

### BRIEF DESCRIPTION OF THE DRAWINGS

FIG. 1 shows data indicating the compound of Formula I and AMG 510 combine synergistically to inhibit cellular proliferation in KRAS G12C mutation in NCI-H358 cells.

FIG. 2A shows a plot of percent activity versus inhibitor concentration (log M) in NCI-H358 cells treated with the compound of Formula I alone and in combination with AMG 510. Tabulated IC50 data in NCI-H358 cells treated with the compound of Formula I alone and in combination with AMG 510.

FIG. 2B shows a bar graph of percent CTG activity that indicates AMG 510 (1 nM) alone did not decrease cell viability in NCI-H358 cells.

FIG. 3 shows data indicating the compound of Formula I and AMG 510 combine synergistically to inhibit cellular proliferation in KRAS G12C mutated NCI-H2122.

FIG. 4A shows a plot of percent activity versus inhibitor concentration (log M) in NCI-H2122 cells treated with the compound of Formula I alone and in combination with various concentrations of AMG 510.

FIG. 4B shows a bar graph of percent CTG activity that indicates AMG 510 (1 nM) alone did not decrease cell viability in NCI-H2122 cells.

FIG. 5 shows a matrix representation of HSA synergy and antagonism, indicating the compound of Formula I and adagrasib combine synergistically to inhibit cellular proliferation in KRAS G12C mutated NCI-H358 cells.

FIG. 6A shows a plot of percent activity versus inhibitor concentration (log M) in NCI-H358 cells treated with the compound of Formula I alone (solid circles) and in combination (solid squares) with 1 nM of adagrasib.

FIG. 6B shows a bar graph of percent CTG activity that indicates adagrasib alone at 1 nM did not decrease cell viability in NCI-H358 cells

FIG. 7 shows a matrix representation of HSA synergy and antagonism, indicating the compound of Formula I and adagrasib combine synergistically to inhibit cellular proliferation in KRAS G12C mutated NCI-H2122 cells.

FIG. 8 shows a matrix representation of HSA synergy and antagonism, indicating the compound of Formula I and adagrasib combine synergistically to inhibit cellular proliferation in KRAS G12C mutated KYSE-410 cells.

FIG. 9A shows a plot of percent activity versus inhibitor concentration (log M) in NCI-H2122 cells treated with the compound of Formula I alone (solid circles, Line 1) and in combination with 1 nM (solid squares, Line 2), 5 nM (solid circles, Line 3), or 10 nM (solid squares, Line 4) of adagrasib.

FIG. 9B shows a bar graph of percent CTG activity indicating that 1 nM, 5 nM or 10 nM of adagrasib alone did not decrease cell viability in NCI-H2122 cells.

FIG. 10A shows a plot of tumor volume (mm³) versus treatment period (days) for a KRAS G12C mutated CRC022 PDX tumor xenograft model treated with vehicle (solid circles, Line 1), adagrasib alone (30 mg/kg QD, solid triangles, Line 2), the compound of Formula I alone at 10 mg/kg/dose BID (solid circles, Line 3), the compound of Formula I at 30 mg/kg QD dose (solid triangles, Line 4), the combination of the compound of Formula I (10 mg/kg/dose BID) and adagrasib at 30 mg/kg QD (solid circles, Line 5), and the combination of the compound of Formula I (30 mg/kg/QD) and adagrasib at 30 mg/kg QD (solid triangles, Line 6).

FIG. 10B shows a plot of tumor volume (mm³) versus treatment period (days) for a KRAS G12C mutated H2122 CDX tumor xenograft model treated with vehicle (solid circles, Line 1), adagrasib alone (30 mg/kg QD, solid triangles, Line 2), the compound of Formula I alone (10 mg/kg/dose BID, solid circles, Line 3), the compound of Formula I alone at 30 mg/kg QD (solid triangles, Line 4), the combination of the compound of Formula I (10 mg/kg/dose BID) and adagrasib at 30 mg/kg QD (solid circles, Line 5), and the combination of the compound of Formula I (30 mg/kg/QD) and adagrasib at 30 mg/kg QD (solid triangles, Line 6).

FIG. 11 shows a graph of tumor volume over a period of treatment time with a regimen of the compound of Formula I alone, sotorasib alone, and the combination of the compound of Formula I and sotorasib in KRAS G12C mutant NSCLC CDX model SW1573.

FIG. 12 shows a graph of tumor volume over a period of treatment time with a regimen of the compound of Formula I alone, sotorasib alone, and the combination of the compound of Formula I and sotorasib in KRAS G12C mutant NSCLC CDX model NCI-H358.

FIG. 13 shows a graph of tumor volume over a period of treatment time with a regimen of the compound of Formula I alone, sotorasib alone, and the combination of the compound of Formula I and sotorasib in KRAS G12C mutant esophageal squamous cell carcinoma CDX model KYSE-410.

FIG. 14 shows a graph of tumor volume over a period of treatment time with a regimen of the compound of Formula I alone, sotorasib alone, and the combination of the compound of Formula I and sotorasib in KRAS G12C mutant CRC PDX model CO-04-0310.

FIG. 15 shows a graph of tumor volume over a period of treatment time with a regimen of the compound of Formula I alone, sotorasib alone, and the combination of the compound of Formula I and sotorasib in KRAS G12C mutant CRC PDX model CR2528.

FIG. 16A shows a graph of tumor volume over a period of treatment time with a regimen of the compound of Formula I alone (30 mg/kg QD), sotorasib (100 mg/kg QD) alone, and the combination of the compound of Formula I (30 mg/kg QD) and sotorasib (100 mg/kg QD) in KRAS G12C mutant NSCLC CDX model NCI-H2122.

FIG. 16B shows a plot of tumor volume versus treatment period (days) for a KEAP1 mutant and KRAS G12C mutant NSCLC CDX model NCI-H2122 tumor xenograft model treated with vehicle (solid circles, Line 1), sotorasib alone (100 mg/kg QD, solid circles, Line 2), the compound of Formula I alone (10 mg/kg/dose BID, solid circles, Line 3), and the combination of the compound of Formula I (10 mg/kg/dose BID) and sotorasib (100 mg/kg QD, solid circles, Line 4).

FIG. 17 shows a graph of tumor volume over a period of treatment time with a regimen of the compound of Formula I alone, sotorasib alone, and the combination of the compound of Formula I and sotorasib in KRAS G12C mutant CRC PDX model CRC022.

### DETAILED DESCRIPTION OF THE INVENTION

### I. General

The present embodiments provide methods of treating a subject having cancer comprising administering to the subject a therapeutically effective amount of a compound of Formula I or its pharmaceutically acceptable salt:

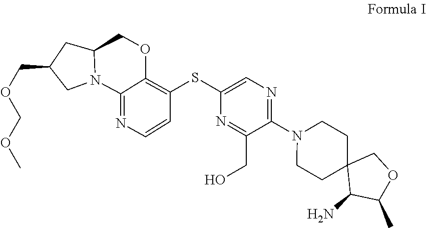 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an inhibitor of KRAS having a G12C mutation. The Examples below indicate a synergy for the combination that was unexpected. The combination therapies disclosed herein, employing the compound of Formula I or its pharmaceutically acceptable salt, can exhibit superior results compared to combinations of alternative SHP2 inhibitors used in combination with inhibitors of KRAS bearing the G12C mutation. Moreover, the combinations of the SHP2 inhibitor compound of Formula I and inhibitors of KRAS G12C provide methods that allow the use of lower dosages of either agent used alone in a monotherapy, which can aid in reducing potential side effects. In particular, the combination therapies can be effective in cancer cells that express the G12C mutation. Accordingly, such treatments comport with the use of companion diagnostics to aid in proper patient population selection. These and other advantages will be recognized by those skilled in the art.

Human KRAS G12C mutated tumors retained significant intrinsic nucleotide cycling between its active state (GTP-bound) and inactive state (GDP-bound). The KRAS G12C inhibitors (G12Ci) showed promising activity by binding to the inactive state (GDP-bound) of KRAS and preventing its reactivation via nucleotide exchange. Negative feedback activation of RTKs and one of their downstream mediator proteins, SHP2, acted as a potential adaptive resistance mechanism. SHP2 was required for guanine nucleotide cycling and its activity promoted growth in KRAS G12C tumors.

### II. Definitions

Unless specifically indicated otherwise, all technical and scientific terms used herein have the same meaning as commonly understood by those of ordinary skill in the art to which the embodiments are directed. In addition, any method or material similar or equivalent to a method or material described herein can be used in the practice of the embodiments herein. For purposes of the embodiments disclosed herein, the following terms are defined.

“A,” “an,” or “the” as used herein not only include aspects with one member, but also include aspects with more than one member. For instance, the singular forms “a,” “an,” and “the” include plural referents unless the context clearly dictates otherwise. Thus, for example, reference to “a cell” includes a plurality of such cells and reference to “the agent” includes reference to one or more agents known to those skilled in the art, and so forth.

“Pharmaceutically acceptable excipient” refers to a substance that aids the administration of an active agent to and absorption by a subject. Pharmaceutical excipients useful in the present embodiments include, but are not limited to, binders, fillers, disintegrants, lubricants, surfactants, coatings, sweeteners, flavors and colors. One of skill in the art will recognize that other pharmaceutical excipients are useful in the present embodiments.

“Treat”, “treating” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

“Administering” refers to oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject. In the context of the combination therapies disclosed herein, administration can be at separate times or simultaneous or substantially simultaneous.

“Co-administering” or “administering in combination with” as used herein refers to administering a composition described herein at the same time, just prior to, or just after the administration of one or more additional therapies. The compounds provided herein can be administered alone or can be co-administered to the patient. Coadministration is meant to include simultaneous or sequential administration of the compounds individually or in combination (more than one compound). Coadministration is meant to include administration of the compounds on the same day, within the same week, and/or within the same treatment schedule. Compounds may have different administration schedules but still be co-administered if they are administered within the same treatment schedule. For example, palbociclib may be administered once a day for three weeks within a four-week treatment schedule, and the compound of Formula I is co-administered with palbociclib if it is administered at any time within the four-week treatment schedule.

“Therapeutically effective amount” refers to a dose that produces therapeutic effects for which it is administered. The exact dose will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins), each of which is incorporated herein by reference in its entirety for all of its teachings, including without limitation all methods, compounds, compositions, data and the like, for use with any of the embodiments and disclosure herein. In sensitized cells, the therapeutically effective dose can often be lower than the conventional therapeutically effective dose for non-sensitized cells.

“Inhibition,” “inhibits” and “inhibitor” refer to a compound that partially or completely blocks or prohibits or a method of partially or fully blocking or prohibiting, a specific action or function.

“Cancer” refers to all types of cancer, neoplasm or malignant tumors found in mammals (e.g. humans), including, without limitation, leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, pancreatic ductal adenocarcinoma (PDAC), skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

“KRAS G12C inhibitor” refers generally to any inhibitor of KRAS bearing the G12C mutation. Such inhibitors include, those known in the art that covalently bind to the 12-cysteine residue, such as AMG 510 (Amgen) and MRTX849 (Mirati). Other examples of KRAS G12C inhibitors are disclosed in pending U.S. Provisional Application Nos. 63/082,221 (TRICYCLIC PYRIDONES AND PYRIMIDONES filed 23 Sep. 2020) and 63/116,146 (PYRROLIDINE-FUSED HETEROCYCLES filed 19 Nov. 2020), each of which are incorporated herein by reference in their entirety. In some embodiments, one or more of the inhibitors listed in this paragraph and elsewhere herein, and those in the incorporated applications, can be specifically excluded from one or more of the embodiments set forth herein, including without limitation, any methods, kits and compositions of matter, etc.

“Subject” refers to a living organism suffering from or prone to a disease or condition that can be treated by administration of a pharmaceutical composition as provided herein. Non-limiting examples include humans, other mammals, bovines, rats, mice, dogs, monkeys, goat, sheep, cows, deer, horse, and other non-mammalian animals. In some embodiments, the patient is human.

### III. Dosing Methods

In some embodiments, the compound of Formula I, or a pharmaceutically acceptable salt thereof, is formulated as a pharmaceutical composition. In some embodiments, the compound of Formula I, or a pharmaceutically acceptable salt thereof, is formulated as an oral composition.

In some embodiments, the compound of Formula I, or a pharmaceutically acceptable salt thereof, is administered once or twice a day. In some embodiments, the compound of Formula I, or a pharmaceutically acceptable salt thereof, is administered once a day. In some embodiments, the compound of Formula I, or a pharmaceutically acceptable salt thereof, is administered twice a day. In some embodiments, the compound of Formula I, or a pharmaceutically acceptable salt thereof, is administered over a continuous 28-day cycle.

In some embodiments, the compound of Formula I, or a pharmaceutically acceptable salt thereof, is administered once a day in the amount of about 10 mg to about 140 mg.

In some embodiments, the compound of Formula I, or a pharmaceutically acceptable salt thereof, is administered once a day for a 3-week cycle, comprising 2 weeks of administration of the compound followed by 1 week of no administration of the compound.

In some embodiments, the compound of Formula I, or a pharmaceutically acceptable salt thereof, is administered once a day for a 4-week cycle, comprising 3 weeks of administration of the compound followed by 1 week of no administration of the compound.

In some embodiments, the compound of Formula I, or a pharmaceutically acceptable salt thereof, is administered over a period of 6 weeks. In some embodiments, the compound of Formula I, or a pharmaceutically acceptable salt thereof, is administered over a period of 8 weeks.

In some embodiments, the compound of Formula I, or a pharmaceutically acceptable salt thereof, is administered 3 times a week. In some embodiments, the compound of Formula I, or a pharmaceutically acceptable salt thereof, is administered on day 1, day 3, and day 5 of the week.

In some embodiments, the compound of Formula I, or a pharmaceutically acceptable salt thereof, is administered 4 times a week.

In some embodiments, the compound of Formula I, or a pharmaceutically acceptable salt thereof, is administered for a 3-week cycle, comprising 2 weeks of administration of the compound followed by 1 week of no administration of the compound.

In some embodiments, the compound of Formula I, or a pharmaceutically acceptable salt thereof, is administered for a 4-week cycle, comprising 3 weeks of administration of the compound followed by 1 week of no administration of the compound.

In some embodiments, the compound of Formula I, or a pharmaceutically acceptable salt thereof, is administered twice a day, two days per week. In some embodiments, the compound of Formula I, or a pharmaceutically acceptable salt thereof, is administered over a period of 8 weeks. In some embodiments, the compound of Formula I, or a pharmaceutically acceptable salt thereof, is administered on day 1 and day 2 of each week.

In some embodiments, the cancer is selected from lung cancer, stomach cancer, liver cancer, colon cancer, kidney cancer, breast cancer, pancreatic cancer, pancreatic ductal adenocarcinoma (PDAC), juvenile myelomonocytic leukemia, neuroblastoma, melanoma, and acute myeloid leukemia.

### IV. Combination Methods

In another aspect, the present disclosure provides a method of treating a subject having cancer comprising administering to the subject a therapeutically effective amount of a compound of Formula I or its pharmaceutically acceptable salt:

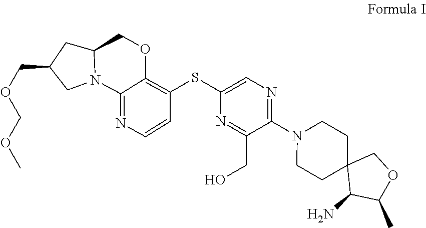 [SMILES: COCOC[C@H]1C[C@H]2COc3c(Sc4cnc(N5CCC6(CC5)CO[C@@H](C)[C@H]6N)c(CO)n4)ccnc3N2C1]

in combination with an inhibitor of KRAS G12C. As disclosed herein, a significant synergy was observed beyond that which had been anticipated for such a combination administration. Any suitable inhibitor can be used, including any disclosed herein. Examples include, but are not limited to, AMG 510 (sotorasib, LUMAKRAS™), MRTX849 (adagrasib), ARS-3248, GDC-6036, BI 1701963, tipifarnib and BBP-454. In some embodiments, one or more of the inhibitors listed in this paragraph and elsewhere herein can be specifically excluded from the embodiments set forth herein, including without limitation, any methods, kits and compositions of matter, etc.

In some embodiments, the methods disclosed herein are suitable for the treatment of any cancer in which there is a KRAS G12C mutation. In some embodiments, the is cancer colorectal cancer. In some embodiments, the cancer is ovarian cancer. In some embodiments, the cancer is pancreatic cancer. In some embodiments, the cancer is pancreatic ductal adenocarcinoma (PDAC). In some embodiments, the cancer is non-small cell lung cancer (NSCLC). In some embodiments, the cancer is cholangiocarcinoma. As will be appreciated by those skilled in the art, tumors may metastasize from a first or primary locus of tumor to one or more other body tissues or sites. In particular, metastases to the central nervous system (i.e., secondary CNS tumors), and particularly the brain (i.e., brain metastases), are well documented for tumors and cancers, such as breast, lung, melanoma, renal and colorectal. As such, the methods disclosed herein can be used for the treatment of metastases (i.e., metastatic tumor growth) to other organs as well.

In some embodiments, the method comprises administering a third MAPK pathway inhibitor. Without being bound by theory, suppression of MAPK signaling in cancer cells can result in downregulation of PD-L1 expression and increase the likelihood that the cancer cells are detected by the immune system. Such third MAPK pathway inhibitors may be based on other mutations of proteins in the MAPK pathway. In some embodiments, any MAPK pathway inhibitor can be employed, including those targeting KRAS, NRAS, HRAS, PDGFRA, PDGFRB, MET, FGFR, ALK, ROS1, TRKA, TRKB, TRKC, EGFR, IGFIR, GRB2, SOS, ARAF, BRAF, RAF1, MEK1, MEK2, c-Myc, CDK4, CDK6, CDK2, ERK1, and ERK2. Exemplary MAPK pathway inhibitors include, without limitation, afatinib, osimertinib, erlotinib, gefitinib, lapatinib, neratinib, dacomitinib, vandetanib, cetuximab, panitumumab, nimotuzumab, necitumumab, trametinib, binimetinib, cobimetinib, selumetinib, ulixertinib, LTT462, and LY3214996. In some embodiments, one or more of the above-listed inhibitors can be specifically excluded from the embodiments set forth herein, including without limitation, any methods, kits and compositions of matter, etc.

The methods disclosed herein can be combined with other chemotherapeutic agents. Examples of such agents can be found in Cancer Principles and Practice of Oncology by V. T. Devita and S. Hellman (editors), 6th edition (Feb. 15, 2001), Lippincott Williams & Wilkins Publishers; which is incorporated herein by reference in its entirety for all of its teachings, including without limitation all methods, compounds, compositions, data and the like, for use with any of the embodiments and disclosure herein. A person of ordinary skill in the art would be able to discern which combinations of agents would be useful based on the particular characteristics of the drugs and the disease involved.

In some embodiments, the methods can include the co-administration of at least one cytotoxic agent. The term “cytotoxic agent” as used herein refers to a substance that inhibits or prevents a cellular function and/or causes cell death or destruction. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof.

Examples of cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signaling inhibitors; HDAC inhibitors, proteasome inhibitors; and inhibitors of cancer metabolism.

Chemotherapeutic agents include chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitinib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNE®, Wyeth), Lapatinib (TYKERB®, GSK572016, Glaxo Smith Kline), Lonafarnib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC 1065 (including its adozelesin, carzelesin and bizelesin synthetic analogs); cryptophycins (particularly cryptophycin 1 and cryptophycin 8); adreno corticosteroids (including prednisone and prednisolone); cyproterone acetate; 5-alpha-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6 azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4 (5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and HRAS; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1 λ antibody genetically modified to recognize interleukin-12 p40 protein.

Chemotherapeutic agent also includes “EGFR inhibitors,” which refers to compounds that bind to or otherwise interact directly with EGFR or its mutant forms and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A: 636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279 (29): 30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl) propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy) quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl) methoxy]phenyl]-6 [5[[[2methylsulfonyl)ethyl]amino]methyl]-2-furanyl]-4-quinazolinamine). Each of the above-described references is incorporated herein by reference in its entirety for all of its teachings, including without limitation all methods, compounds, compositions, data and the like, for use with any of the embodiments and disclosure herein.

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d]pyrimidines; curcumin (diferuloyl methane, 4,5-bis(4-fluoroanilino) phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca). Each of the above-described references is incorporated herein by reference in its entirety for all of its teachings, including without limitation all methods, compounds, compositions, data and the like, for use with any of the embodiments and disclosure herein.

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN Bio Therapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFα) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-MI prime; Secreted homotrimeric LTa3 and membrane bound heterotrimer LTa1/β2 blockers such as Anti-lymphotoxin alpha (LTa); radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); miscellaneous investigational agents such as thioplatin, PS-341, phenylbutyrate, ET-18-OCH₃, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam andisoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

In certain embodiments, chemotherapeutic agents include, but are not limited to, doxorubicin, dexamethasone, vincristine, cyclophosphamide, fluorouracil, topotecan, interferons, platinum derivatives, taxanes (e.g., paclitaxel, docetaxel), vinca alkaloids (e.g., vinblastine), anthracyclines (e.g., doxorubicin), epipodophyllotoxins (e.g., etoposide), cisplatin, an mTOR inhibitor (e.g., a rapamycin), methotrexate, actinomycin D, dolastatin 10, colchicine, trimetrexate, metoprine, cyclosporine, daunorubicin, teniposide, amphotericin, alkylating agents (e.g., chlorambucil), 5-fluorouracil, campthothecin, cisplatin, metronidazole, and imatinib mesylate, among others. In other embodiments, a compound disclosed herein is administered in combination with a biologic agent, such as bevacizumab or panitumumab.

In certain embodiments, compounds disclosed herein, or a pharmaceutically acceptable composition thereof, are administered in combination with an antiproliferative or chemotherapeutic agent selected from any one or more of abarelix, aldesleukin, alemtuzumab, alitretinoin, allopurinol, altretamine, amifostine, anastrozole, arsenic trioxide, asparaginase, azacitidine, BCG live, bevacuzimab, fluorouracil, bexarotene, bleomycin, bortezomib, busulfan, calusterone, capecitabine, camptothecin, carboplatin, carmustine, cetuximab, chlorambucil, cladribine, clofarabine, cyclophosphamide, cytarabine, dactinomycin, darbepoetin alfa, daunorubicin, denileukin, dexrazoxane, docetaxel, doxorubicin (neutral), doxorubicin hydrochloride, dromostanolone propionate, epirubicin, epoetin alfa, elotinib, estramustine, etoposide phosphate, etoposide, exemestane, filgrastim, floxuridine, fludarabine, fulvestrant, gefitinib, gemcitabine, gemtuzumab, goserelin acetate, histrelin acetate, hydroxyurea, ibritumomab, idarubicin, ifosfamide, imatinib mesylate, interferon alfa-2a, interferon alfa-2b, irinotecan, lenalidomide, letrozole, leucovorin, leuprolide acetate, levamisole, lomustine, megestrol acetate, melphalan, mercaptopurine, 6-MP, mesna, methotrexate, methoxsalen, mitomycin C, mitotane, mitoxantrone, nandrolone, nelarabine, nofetumomab, oprelvekin, oxaliplatin, paclitaxel, palifermin, pamidronate, pegademase, pegaspargase, pegfilgrastim, pemetrexed disodium, pentostatin, pipobroman, plicamycin, porfimer sodium, procarbazine, quinacrine, rasburicase, rituximab, sargramostim, sorafenib, streptozocin, sunitinib maleate, talc, tamoxifen, temozolomide, teniposide, VM-26, testolactone, thioguanine, 6-TG, thiotepa, topotecan, toremifene, tositumomab, trastuzumab, tretinoin, ATRA, uracil mustard, valrubicin, vinblastine, vincristine, vinorelbine, zoledronate, or zoledronic acid.

In some embodiments, the dosing of the compound of Formula I can be in any suitable amount to treat the cancer. For example, the dosing could be a daily dosage of between 1 mg weight up to 500 mg. As an additional example, the daily dose could be in a range from about 20 mg to 400 mg (or any sub-range or sub-value there between, including endpoints). In some embodiments, the range of dosing of the compound of Formula I can be from 10 mg to 300 mg. In some embodiments, the range of dosing of the compound of Formula I can be from 10 mg to 100 mg. In some embodiments, the range of dosing of the compound of Formula I can be from 5 mg to 50 mg. The daily dosage can be achieved by administering a single administered dosage (e.g., QD) or via multiple administrations during a day (e.g., BID, TID, QID, etc.) to provide the total daily dosage. In some embodiments, the dosing of the KRAS inhibitor is any suitable amount. For example, it can be an amount in a range from 1 mg to 1,000 mg daily (or any sub-range or sub-value there between, including endpoints). Dosing of the KRAS inhibitor may be the same or less than the approved dosing for any given KRAS inhibitor and may depend on a given indication. For example, AMG 510 may be administered in a range from 500 mg to 1,000 mg once daily. For example, MRTX849 may be administered in a range from 500 mg to 1200 mg once daily. It will be appreciated that each of the recited ranges above can include any sub-range or sub-point therein, inclusive of endpoints. It will be appreciated that each of the recited ranges above can include any sub-range or sub-point therein, inclusive of endpoints. A common dose range for adult humans is generally from 5 mg to 2 g/day. Tablets or other forms of presentation provided in discrete units may conveniently contain an amount of one or more compounds which is effective at such dosage or as a multiple of the same, for instance, units containing 5 mg to 500 mg, usually around 10 mg to 200 mg. The amount of active ingredient that may be combined with the carrier materials to produce a single dosage form will vary depending upon the host treated and the particular mode of administration. In some embodiments, the administration is oral.

In some embodiments, there are provided methods of treating lung or esophageal cancer in a subject comprising orally administering to the subject a therapeutically effective amount of a compound of Formula I or its pharmaceutically acceptable salt in combination with AMG 510. In some embodiments, the compound of Formula I is administered once or twice daily. In some embodiments, AMG 510 is administered once or twice daily. The drugs can be co-administered as described herein, for example.

In some embodiments, there are provided methods of treating lung, colorectal, esophageal or breast cancer in a subject comprising orally administering to the subject a therapeutically effective amount of a compound of Formula I or its pharmaceutically acceptable salt in combination with adagrasib. In some embodiments, the compound of Formula Iis administered once or twice daily. In some embodiments, adagrasib is administered once or twice daily. The drugs can be co-administered as described herein, for example.

In some embodiments, the subject is a human. In some embodiments, the subject is a mammal other than a human, such as a primate, a rodent, a dog, a cat, or other small animal.

### Compositions

The compound of Formula I disclosed herein may exist as salts. The present embodiments include such salts, which can be pharmaceutically acceptable salts. Examples of applicable salt forms include hydrochlorides, hydrobromides, sulfates, methanesulfonates, nitrates, maleates, acetates, citrates, fumarates, tartrates (eg (+)-tartrates, (−)-tartrates or mixtures thereof including racemic mixtures, succinates, benzoates and salts with amino acids such as glutamic acid. These salts may be prepared by methods known to those skilled in art. Also included are base addition salts such as sodium, potassium, calcium, ammonium, organic amino, or magnesium salt, or a similar salt. When compounds of the present embodiments contain relatively basic functionalities, acid addition salts can be obtained by contacting the neutral form of such compounds with a sufficient amount of the desired acid, either neat or in a suitable inert solvent. Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like. Certain specific compounds of the present embodiments contain both basic and acidic functionalities that allow the compounds to be converted into either base or acid addition salts.

Other salts include acid or base salts of the compounds used in the methods of the present embodiments. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, and quaternary ammonium (methyl iodide, ethyl iodide, and the like) salts. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference in its entirety for all of its teachings, including without limitation all methods, compounds, compositions, data and the like, for use with any of the embodiments and disclosure herein.

Pharmaceutically acceptable salts include salts of the active compounds which are prepared with relatively nontoxic acids or bases, depending on the particular substituents found on the compounds described herein. When compounds of the present embodiments contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present embodiments contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19), which is incorporated herein by reference in its entirety for all of its teachings, including without limitation all methods, compounds, compositions, data and the like, for use with any of the embodiments and disclosure herein. Certain specific compounds of the present embodiments contain both basic and acidic functionalities that allow the compounds to be converted into either base or acid addition salts.

The neutral forms of the compounds are preferably regenerated by contacting the salt with a base or acid and isolating the parent compound in the conventional manner. The parent form of the compound differs from the various salt forms in certain physical properties, such as solubility in polar solvents.

Certain compounds of the present embodiments can exist in unsolvated forms as well as solvated forms, including hydrated forms. In general, the solvated forms are equivalent to unsolvated forms and are encompassed within the scope of the present embodiments. Certain compounds of the present embodiments may exist in multiple crystalline or amorphous forms. In general, all physical forms are equivalent for the uses contemplated by the present embodiments and are intended to be within the scope of the present embodiments.

Certain compounds of the present embodiments possess asymmetric carbon atoms (optical centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present embodiments. The compounds of the present embodiments do not include those which are known in art to be too unstable to synthesize and/or isolate. The present embodiments are meant to include compounds in racemic and optically pure forms. Optically active (R)- and (S)-, or (D)- and (L)-isomers may be prepared using chiral synthons or chiral reagents, or resolved using conventional techniques.

Unless otherwise stated, the compounds of the present embodiments may also contain unnatural proportions of atomic isotopes at one or more of the atoms that constitute such compounds. For example, the compounds of the present embodiments may be labeled with radioactive or stable isotopes, such as for example deuterium (²H), tritium (³H), iodine-125 (¹²⁵I), fluorine-18 (¹⁸F), nitrogen-15 (¹⁵N), oxygen-17 (¹⁷O), oxygen-18 (¹⁸O), carbon-13 (¹³C), or carbon-14 (¹⁴C). All isotopic variations of the compounds of the present embodiments, whether radioactive or not, are encompassed within the scope of the present embodiments.

In addition to salt forms, the present embodiments provide compounds, which are in a prodrug form. Prodrugs of the compounds described herein are those compounds that readily undergo chemical changes under physiological conditions to provide the compounds of the present embodiments. Additionally, prodrugs can be converted to the compounds of the present embodiments by chemical or biochemical methods in an ex vivo environment. For example, prodrugs can be slowly converted to the compounds of the present embodiments when placed in a transdermal patch reservoir with a suitable enzyme or chemical reagent.

In some embodiments, there are provided pharmaceutical compositions comprising the compound of Formula I and a pharmaceutically acceptable excipient. In some embodiments, the pharmaceutical compositions are configured as an oral tablet preparation.

The compounds of the present embodiments can be prepared and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. The compounds of the present embodiments can also be administered by injection, that is, intravenously, intramuscularly, intracutaneously, subcutaneously, intraduodenally, or intraperitoneally. Also, the compounds described herein can be administered by inhalation, for example, intranasally. Additionally, the compounds of the present embodiments can be administered transdermally. The compound of Formula I disclosed herein can also be administered by in intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995), which is incorporated herein by reference in its entirety for all of its teachings, including without limitation all methods, compounds, compositions, data and the like, for use with any of the embodiments and disclosure herein. Accordingly, the present embodiments also provide pharmaceutical compositions including one or more pharmaceutically acceptable carriers and/or excipients and either a compound of Formula I, or a pharmaceutically acceptable salt of a compound of Formula I.

For preparing pharmaceutical compositions from the compounds of the present embodiments, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, surfactants, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”), which is incorporated herein by reference in its entirety for all of its teachings, including without limitation all methods, compounds, compositions, data and the like, for use with any of the embodiments and disclosure herein.

In powders, the carrier is a finely divided solid, which is in a mixture with the finely divided active component. In tablets, the active component is mixed with the carrier having the necessary binding properties and additional excipients as required in suitable proportions and compacted in the shape and size desired.

The powders, capsules and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers including, but not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations disclosed herein can also be used orally using, for example, push-fit capsules made of gelatin, as well as soft, sealed capsules made of gelatin and a coating such as glycerol or sorbitol. Push-fit capsules can contain the compounds of Formula I mixed with a filler or binders such as lactose or starches, lubricants such as talc or magnesium stearate, and, optionally, stabilizers. In soft capsules, the compounds of Formula I may be dissolved or suspended in suitable liquids, such as fatty oils, liquid paraffin, or liquid polyethylene glycol with or without stabilizers.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the active component in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of Formula I in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997, which is incorporated herein by reference in its entirety for all of its teachings, including without limitation all methods, compounds, compositions, data and the like, for use with any of the embodiments and disclosure herein. The pharmaceutical formulations disclosed herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The pharmaceutical formulations of the compound of Formula I disclosed herein can be provided as a salt and can be formed with bases, namely cationic salts such as alkali and alkaline earth metal salts, such as sodium, lithium, potassium, calcium, magnesium, as well as ammonium salts, such as ammonium, trimethyl-ammonium, diethylammonium, and tris-(hydroxymethyl)-methyl-ammonium salts.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

The dosage regimen also takes into consideration pharmacokinetics parameters well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra; each of which is incorporated herein by reference in its entirety for all of its teachings, including without limitation all methods, compounds, compositions, data and the like, for use with any of the embodiments and disclosure herein). The state of the art allows the clinician to determine the dosage regimen for each individual patient, GR and/or MR modulator and disease or condition treated.

Single or multiple administrations of the compound of Formula I formulations can be administered depending on the dosage and frequency as required and tolerated by the patient. The formulations should provide a sufficient quantity of active agent to effectively treat the disease state. Thus, in one embodiment, the pharmaceutical formulations for oral administration of the compound of Formula I is in a daily amount of between about 0.5 to about 30 mg per kilogram of body weight per day, including all sub-ranges and sub-values therein, inclusive of endpoints. In an alternative embodiment, dosages are from about 1 mg to about 20 mg per kg of body weight per patient per day are used. Lower dosages can be used, particularly when the drug is administered to an anatomically secluded site, such as the cerebral spinal fluid (CSF) space, in contrast to administration orally, into the blood stream, into a body cavity or into a lumen of an organ. Substantially higher dosages can be used in topical administration. Actual methods for preparing formulations including the compound of Formula I for parenteral administration are known or apparent to those skilled in the art and are described in more detail in such publications as Remington's, supra. See also Nieman, In “Receptor Mediated Antisteroid Action,” Agarwal, et al., eds., De Gruyter, New York (1987), which is incorporated herein by reference in its entirety for all of its teachings, including without limitation all methods, compounds, compositions, data and the like, for use with any of the embodiments and disclosure herein.

In some embodiments, co-administration includes administering one active agent within 0.5, 1, 2, 4, 6, 8, 10, 12, 16, 20, or 24 hours (or any sub-range of time or sub-value of time within a 24 hour period) of a second active agent. Co-administration includes administering two active agents simultaneously, approximately simultaneously (e.g., within about 1, 5, 10, 15, 20, or 30 minutes of each other (or any sub-range of time or sub-value of time from 0-30 minutes for example)), or sequentially in any order. In some embodiments, co-administration can be accomplished by co-formulation, i.e., preparing a single pharmaceutical composition including both active agents. In some embodiments, the active agents can be formulated separately. In some embodiments, the active and/or adjunctive agents may be linked or conjugated to one another. At least one administered dose of drugs can be administered, for example, at the same time. At least one administered dose of the drugs can be administered, for example, within minutes or less than an hour of each other. At least one administered dose of drugs can be administered, for example, at different times, but on the same day, or on different days.

After a pharmaceutical composition including a compound of Formula I disclosed herein has been formulated in one or more acceptable carriers, it can be placed in an appropriate container and labeled for treatment of an indicated condition. For administration of the compounds of Formula I, such labeling would include, e.g., instructions concerning the amount, frequency and method of administration.

### Pharmaceutical Dosing

The dosage regimen for the compounds herein will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A clinical practitioner can determine and prescribe the effective amount of the drug required to prevent, counter, or arrest the progress of the disease or disorder.

By way of general guidance, the daily oral dosage of each active ingredient, when used for the indicated effects, will range between about 0.001 to about 1000 mg/kg of body weight, preferably between about 0.01 to about 100 mg/kg of body weight per day, and most preferably between about 0.1 to about 20 mg/kg/day. In some embodiments, a compound of Formula (I) may be administered at a dose of between about 10 mg/day and about 200 mg/day. In some embodiments, a compound of Formula (I) may be administered at a dose of about 10 mg/day, 20 mg/day, 30 mg/day, 40 mg/day, 50 mg/day, 60 mg/day, 70 mg/day, 80 mg/day, 90 mg/day, 100 mg/day, 110 mg/day, 120 mg/day, 130 mg/day, 140 mg/day, 150 mg/day, 160 mg/day, 170 mg/day, 180 mg/day, 190 mg/day, or 200 mg/day. The dose may be any value or subrange within the recited ranges.

Depending on the patient's condition and the intended therapeutic effect, the dosing frequency for the therapeutic agent may vary, for example, from once per day to six times per day. That is, the dosing frequency may be QD, i.e., once per day, BID, i.e., twice per day; TID, i.e., three times per day; QID, i.e., four times per day; five times per day, or six times per day. In another embodiment, dosing frequency may be BIW, i.e., twice weekly, TIW, i.e., three times a week, or QIW, i.e. four times a week.

Depending on the patient's condition and the intended therapeutic effect, the treatment cycle may have a period of time where no therapeutic agent is administered. As used herein, “interval administration” refers to administration of the therapeutic agent followed by void days or void weeks. For example, the treatment cycle may be 3 weeks long which includes 2 weeks of dosing of the therapeutic agent(s) followed by 1 week where no therapeutic agent is administered. In some embodiments, the treatment cycle is 4 weeks long which includes 3 weeks of dosing followed by 1 week where no therapeutic agent is administered.

The term “treatment cycle” as used herein, means a pre-determined period of time for administering the therapeutic agent. Typically, the patient is examined at the end of each treatment cycle to evaluate the effect of the therapy.

In one embodiment, each of the treatment cycle has about 3 or more days. In another embodiment, each of the treatment cycle has from about 3 days to about 60 days. In another embodiment, each of the treatment cycle has from about 5 days to about 50 days. In another embodiment, each of the treatment cycle has from about 7 days to about 28 days. In another embodiment, each of the treatment cycle has 28 days. In one embodiment, the treatment cycle has about 29 days. In another embodiment, the treatment cycle has about 30 days. In another embodiment, the treatment cycle has about 31 days. In another embodiment, the treatment cycle has about a month-long treatment cycle. In another embodiment, the treatment cycle is any length of time from 3 weeks to 8 weeks. In another embodiment, the treatment cycle is any length of time from 3 weeks to 6 weeks. In yet another embodiment, the treatment cycle is 3 weeks. In another embodiment, the treatment cycle is one month. In another embodiment, the treatment cycle is 4 weeks. In another embodiment, the treatment cycle is 5 weeks. In another embodiment, the treatment cycle is 6 weeks. In another embodiment, the treatment cycle is 7 weeks. In another embodiment, the treatment cycle is 8 weeks. The duration of the treatment cycle may include any value or subrange within the recited ranges, including endpoints.

As used herein, the term “co-administration” or “coadministration” refers to administration of (a) an additional therapeutic agent and (b) a compound of Formula (I), or a salt, solvate, ester and/or prodrug thereof, together in a coordinated fashion. For example, the co-administration can be simultaneous administration, sequential administration, overlapping administration, interval administration, continuous administration, or a combination thereof.

In some embodiments, the dosing regimen for a compound of Formula (I) is once daily over a continuous 28-day cycle. In some embodiments, the once daily dosing regimen for a compound of Formula (I) may be, but is not limited to, 20 mg/day, 30 mg/day, 40 mg/day, 50 mg/day, 60 mg/day. Compounds of Formula (I) may be administered anywhere from 20 mg to 60 mg once a day. The dose may be any value or subrange within the recited ranges.

In some embodiments, the dosing regimen for a compound of Formula (I) is twice daily over a continuous 28-day cycle. In some embodiments, the twice daily dosing regimen for a compound of Formula (I) may be, but is not limited to, 10 mg/day, 20 mg/day, 30 mg/day, 40 mg/day, 50 mg/day, 60 mg/day, 70 mg/day, 80 mg/day, 90 mg/day, 100 mg/day. Compounds of Formula (I) may be administered anywhere from 20 mg to 80 mg twice a day. In some embodiments, compounds of Formula (I) may be administered anywhere from 10 mg/day to 100 mg/day. The dose may be any value or subrange within the recited ranges.

In some embodiments, the dosing regimen for a compound of Formula (I) may be once daily, anywhere from 20 mg to 60 mg per day for two weeks, followed by a one week break over a period of 6 weeks (e.g. 2 weeks on, 1 week off). In some embodiments, the dosing regimen for a compound of Formula (I) may be twice daily, anywhere from 10 mg to 100 mg twice a day for two weeks, followed by a one week break over a period of 6 weeks (e.g. 2 weeks on, 1 week off).

In some embodiments, the dosing regimen for a compound of Formula (I) may be once daily, anywhere from 20 mg to 60 mg per day for three weeks, followed by a one week break over a period of 8 weeks (e.g. 3 weeks on, 1 week off). In some embodiments, the dosing regimen for a compound of Formula (I) may be twice daily, anywhere from 10 mg to 100 mg twice a day for three weeks, followed by a one week break over a period of 8 weeks (e.g. 8 weeks on, 1 week off).

In some embodiments, the dosing regimen for a compound of Formula (I) may be twice daily on days 1 and 2, weekly for 8 weeks. In some embodiments, the dosing amount for compounds of Formula (I) may be, but is not limited to, 10 mg, 20 mg, 30 mg, 40 mg, 50 mg, 60 mg, 70 mg, 80 mg, 90 mg, 100 mg.

In some embodiments, the compound of Formula I, or a pharmaceutically acceptable salt thereof, is administered once a day for a 3-week cycle, comprising 2 weeks of administration of the compound followed by 1 week of no administration of the compound.

In some embodiments, the compound of Formula I, or a pharmaceutically acceptable salt thereof, is administered once a day for a 4-week cycle, comprising 3 weeks of administration of the compound followed by 1 week of no administration of the compound.

In some embodiments, the compound of Formula I, or a pharmaceutically acceptable salt thereof, is administered over a period of 6 weeks. In some embodiments, the compound of Formula I, or a pharmaceutically acceptable salt thereof, is administered over a period of 8 weeks.

In some embodiments, the compound of Formula I, or a pharmaceutically acceptable salt thereof, is administered 3 times a week. In some embodiments, the compound of Formula I, or a pharmaceutically acceptable salt thereof, is administered on day 1, day 3, and day 5 of the week.

In some embodiments, the compound of Formula I, or a pharmaceutically acceptable salt thereof, is administered 4 times a week.

In some embodiments, the compound of Formula I, or a pharmaceutically acceptable salt thereof, is administered for a 3-week cycle, comprising 2 weeks of administration of the compound followed by 1 week of no administration of the compound.

In some embodiments, the compound of Formula I, or a pharmaceutically acceptable salt thereof, is administered for a 4-week cycle, comprising 3 weeks of administration of the compound followed by 1 week of no administration of the compound.

In some embodiments, the compound of Formula I, or a pharmaceutically acceptable salt thereof, is administered twice a day, two days per week. In some embodiments, the compound of Formula I, or a pharmaceutically acceptable salt thereof, is administered over a period of 8 weeks. In some embodiments, the compound of Formula I, or a pharmaceutically acceptable salt thereof, is administered on day 1 and day 2 of each week.

When a compound of Formula I is administered multiple times a week, the dose may be administered on any day or combination of days within the week. For example, administration three times per week may include administration on days 1, 3, and 5; days 1, 2, and 3; 1, 3, and 5; and so on. Administration two days per week may include administration on days 1 and 2; days 1 and 3; days 1 and 4; days 1 and 5; days 1 and 6; days 1 and 7; and so on.

### Cancer

In some embodiments, the cancer has a G12C KRAS mutation. In some embodiments, the cancer has a G12D KRAS mutation. In some embodiments, the cancer has a G12R KRAS mutation. In some embodiments, the cancer has a G12S KRAS mutation. In some embodiments, the cancer has a G12V KRAS mutation. In some embodiments, the cancer has a G12W KRAS mutation. In some embodiments, the cancer has a G13D KRAS mutation. In some embodiments, the cancer has a H95D KRAS mutation. In some embodiments, the cancer has a H95Q KRAS mutation. In some embodiments, the cancer has a H95R KRAS mutation. In some embodiments, the cancer has a Q61H KRAS mutation. In some embodiments, the cancer has a G12D KRAS mutation. In some embodiments, the cancer has a Q61K KRAS mutation. In some embodiments, the cancer has a Q61R NRAS mutation. In some embodiments, the cancer has a R68S KRAS mutation.

In some embodiments, the cancer is non-small cell lung cancer (NSCLC). In some embodiments, the NSCLC is a KRAS G12C mutant NSCLC. In some embodiments, the NSCLC is a KRAS G12D mutant NSCLC. In some embodiments, the NSCLC is a KRAS G12S mutant NSCLC. In some embodiments, the NSCLC is a KRAS G12V mutant NSCLC. In some embodiments, the NSCLC is a KRAS G13D mutant NSCLC. In some embodiments, the NSCLC is a KRAS Q61H mutant NSCLC. In some embodiments, the NSCLC is a KRAS Q61K mutant NSCLC. In some embodiments, the NSCLC is a KRAS G12R mutant NSCLC. In some embodiments, the NSCLC is a KRAS G12W mutant NSCLC. In some embodiments, the NSCLC is a KRAS H95D mutant NSCLC. In some embodiments, the NSCLC is a KRAS H95Q mutant NSCLC. In some embodiments, the NSCLC is a KRAS H95R mutant NSCLC. In some embodiments, the NSCLC is a KRAS G12D mutant NSCLC. In some embodiments, the NSCLC is a KRAS R68S mutant NSCLC.

In some embodiments, the cancer is a KRAS-treated G12C NSCLC. In some embodiments, the cancer is a KRAS-treated G12D NSCLC. In some embodiments, the cancer is a KRAS-treated G12S NSCLC. In some embodiments, the cancer is a KRAS-treated G12V NSCLC. In some embodiments, the cancer is a KRAS-treated G13D NSCLC. In some embodiments, the cancer is a KRAS-treated Q61H NSCLC. In some embodiments, the cancer is a KRAS-treated Q61K NSCLC. In some embodiments, the cancer is a NRAS-treated Q61R NSCLC. In some embodiments, the cancer is a KRAS-treated G12R NSCLC. In some embodiments, the cancer is a KRAS-treated G12W NSCLC. In some embodiments, the cancer is a KRAS-treated H95D NSCLC. In some embodiments, the cancer is a KRAS-treated H95Q NSCLC. In some embodiments, the cancer is a KRAS-treated H95R NSCLC. In some embodiments, the cancer is a KRAS-treated G12D NSCLC. In some embodiments, the cancer is a KRAS-treated R68S NSCLC.

In some embodiments, the cancer is colorectal cancer (CRC). In some embodiments, the CRC is a KRAS mutant CRC. In some embodiments, the CRC is a KRAS G12C mutant CRC. In some embodiments, the CRC is a KRAS G12D mutant CRC. In some embodiments, the CRC is a KRAS G12S mutant CRC. In some embodiments, the CRC is a KRAS G12V mutant CRC. In some embodiments, the CRC is a KRAS G13D mutant CRC. In some embodiments, the CRC is a KRAS Q61H mutant CRC. In some embodiments, the CRC is a KRAS Q61K mutant CRC. In some embodiments, the CRC is a NRAS mutant CRC. In some embodiments, the CRC is a NRAS Q61R mutant CRC.

In some embodiments, the cancer has one or more acquired mutations. In some embodiments, the acquired mutation results from a first-line treatment. In some embodiments, the first-line treatment is a KRAS inhibitor. In some embodiments, the KRAS inhibitor is a KRAS G12C inhibitor. In some embodiments, the KRAS G12C inhibitor is adagrasib. In some embodiments, the KRAS G12C inhibitor is sotorasib. In some embodiments, the cancer is a solid tumor cancer. In some embodiments, the cancer is NSCLC.

In some embodiments, the acquired mutation is an acquired KRAS mutation. In some embodiments, the acquired mutation is KRAS G12C. In some embodiments, the acquired mutation is KRAS G12D. In some embodiments, the acquired mutation is KRAS G12R. In some embodiments, the acquired mutation is KRAS G12V. In some embodiments, the acquired mutation is KRAS G12W. In some embodiments, the acquired mutation is KRAS G13D. In some embodiments, the acquired mutation is KRAS H95D. In some embodiments, the acquired mutation is KRAS H95D. In some embodiments, the acquired mutation is KRAS H95Q. In some embodiments, the acquired mutation is KRAS H95R. In some embodiments, the acquired mutation is KRAS Q61H. In some embodiments, the acquired mutation is KRAS R68S.

In some embodiments, the acquired mutation is an acquired MAPK pathway mutation. In some embodiments, the acquired MAPK pathway mutation is MAP2K1 K57N. In some embodiments, the acquired MAPK pathway mutation is MAP2K1 K57T. In some embodiments, the acquired MAPK pathway mutation is CCDC6-RET. In some embodiments, the acquired MAPK pathway mutation is RITI P128L. In some embodiments, the acquired MAPK pathway mutation is PTEN G209V. In some embodiments, the acquired MAPK pathway mutation is BRAF V600E. In some embodiments, the acquired MAPK pathway mutation is MAP2K1 199_K104del. In some embodiments, the acquired MAPK pathway mutation is MAP2K1 K57N. In some embodiments, the acquired MAPK pathway mutation is EML4-ALK. In some embodiments, the acquired MAPK pathway mutation is EGFR A289A. In some embodiments, the acquired MAPK pathway mutation is FGFR3-TACC3. In some embodiments, the acquired MAPK pathway mutation is AKAP9-BRAF. In some embodiments, the acquired MAPK pathway mutation is RAF1-CCDC176. In some embodiments, the acquired MAPK pathway mutation is RAF1-TRAK1. In some embodiments, the acquired MAPK pathway mutation is NRAS Q61K. In some embodiments, the acquired MAPK pathway mutation is MAP2K1 E102_1103DEL. In some embodiments, the acquired MAPK pathway mutation is NRF1-BRAF.

In some embodiments, the acquired mutation is a KRAS G12C reactivation mutation. In some embodiments, the KRAS G12C reactivation mutation is a RKRAS G12C gene amplification. In some embodiments, the KRAS G12C reactivation mutation is a NF1 R22637 (LoF).

In some embodiments, the acquired mutation is a non-G12C activation KRAS mutation. In some embodiments, the non-G12C activation KRAS mutation is KRAS G12D. In some embodiments, the non-G12C activation KRAS mutation is KRAS G12R. In some embodiments, the non-G12C activation KRAS mutation is KRAS G12V. In some embodiments, the non-G12C activation KRAS mutation is KRAS G12W. In some embodiments, the non-G12C activation KRAS mutation is KRAS G13D. In some embodiments, the non-G12C activation KRAS mutation is KRAS Q61H. In some embodiments, the non-G12C activation KRAS mutation is KRAS Q61K.

In some embodiments, the acquired mutation is a sterically hindering KRAS G12C mutation. In some embodiments, the sterically hindering KRAS G12C mutation is KRAS R68S. In some embodiments, the sterically hindering KRAS G12C mutation is KRAS H95D. In some embodiments, the sterically hindering KRAS G12C mutation is KRAS H95Q. In some embodiments, the sterically hindering KRAS G12C mutation is KRAS H95R. In some embodiments, the sterically hindering KRAS G12C mutation is KRAS Y96C.

In some embodiments, the acquired mutation is an RTK activation mutation. In some embodiments, the RTK activation mutation is EGFR A289V. In some embodiments, the RTK activation mutation is RET M918T. In some embodiments, the RTK activation mutation is MET gene amplification. In some embodiments, the RTK activation mutation is EML-ALK. In some embodiments, the RTK activation mutation is CCDC6-RET. In some embodiments, the RTK activation mutation is FGFR3-TACC3.

In some embodiments, the acquired mutation is a downstream RAS/MAPK activation mutation. In some embodiments, the downstream RAS/MAPK activation mutation is BRAF V600E. In some embodiments, the downstream RAS/MAPK activation mutation is MAP2K I99_K104del. In some embodiments, the downstream RAS/MAPK activation mutation is MAP2K1 I99_K104del. In some embodiments, the downstream RAS/MAPK activation mutation is MAP2K1 E102_1103del. In some embodiments, the downstream RAS/MAPK activation mutation is RAF fusion.

In some embodiments, the acquired mutation is a parallel pathway activation mutation. In some embodiments, the parallel pathway activation mutation is PIK3CA H1047R. In some embodiments, the parallel pathway activation mutation is PIK3R1 S361fs. In some embodiments, the parallel pathway activation mutation is PTEN N48K. In some embodiments, the parallel pathway activation mutation is PTEN G209V. In some embodiments, the parallel pathway activation mutation is RIT1 P128L.

### Kits and Products

Some embodiments relate to kits and products that include the compound of Formula I and/or at least on KRAS G12C inhibitor. For example, the kit or product can include a package or container with a compound of Formula I. Such kits and products can further include a product insert or label with approved drug administration and indication information, including how to use the compound of Formula I in combination with an KRAS G12C inhibitor that is separately provided. The kits can be used in the methods of treating cancer as described herein.

In some aspects, the kits or products can include both a compound of Formula I and at least one KRAS G12C inhibitor. In some embodiments, the KRAS G12C inhibitor is AMG 510, for example. In some embodiments, the KRAS G12C inhibitor is MRTX849, for example. Such kits can include one or more containers or packages, which include one or both combination drugs together in a single container and/or package, or in separate packages/containers. In some instances, the two drugs are separately wrapped, but included in a single package, container or box. Such kits and products can further include a product insert or label with approved drug administration and indication information, including how to use the compound of Formula I in combination with an KRAS G12C inhibitor. The kits can be used in the methods of treating cancer as described herein.

### EXAMPLES

### General Procedures

All starting materials and solvents were obtained either from commercial sources or prepared according to the literature citation.

### Example 1

This Example demonstrates the synergistic combination of the compound of Formula I with KRAS G12C inhibitors.

Cellular proliferation assay: The cells (2000 cells per well) were plated onto 96-well plates in 100 μl cell culture medium and treated with the compound of Formula I alone or the compound of Formula I with fixed concentration of AMG 510. At day 5, 50 μl of CellTiter-Glo (CTG) reagent (Promega) was added and the plates were incubated for 10 minutes with gentle shaking. After 10 minutes incubation, the luminescent signal was determined according to the provider's instruction (Promega), and graph was plotted using Prism GraphPad.

Combination cellular proliferation assays: Cells (2000 cells per well) were plated onto 96-well plates in 100 μl cell culture medium. Cells were treated with the compound of Formula I and AMG 510 at concentrations varying from 0 to 10 μM by using the Tecan D300e Digital Dispenser combination matrix protocol. At day 5, 50 μl of CellTiter-Glo (CTG) reagent (Promega) was added and the plates were incubated for 10 minutes with gentle shaking. After 10 minutes of incubation, the luminescent signal was determined according to the provider's instructions (Promega) and combination data was generated by the standard HSA model using Combenefit software. The combination synergy was represented by positive numbers in results table. The negative numbers represent antagonism of the combination.

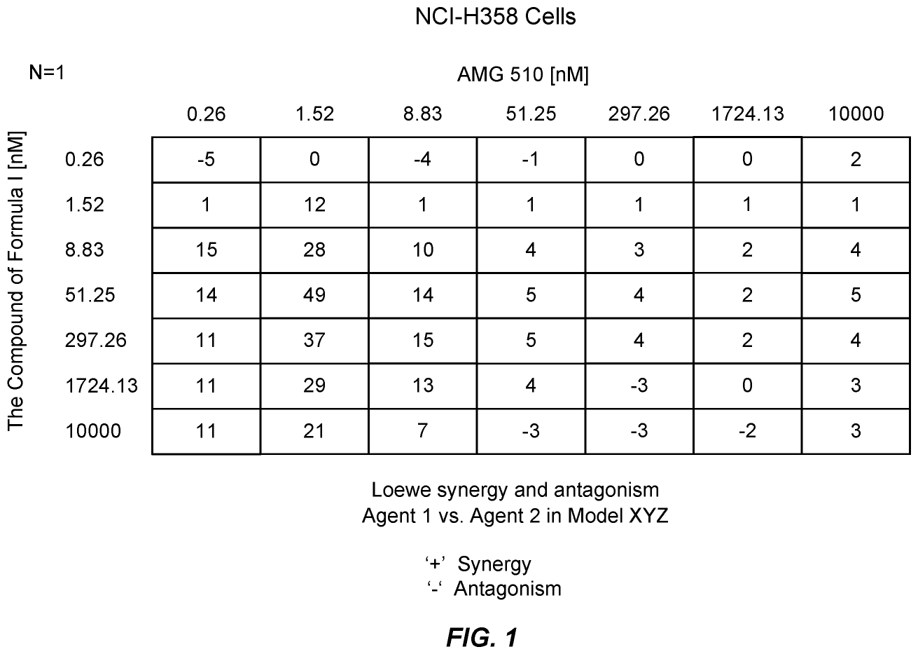

The results of these experiments are indicated in FIGS. 1 to 4 are further discussed below.

FIG. 1 shows data indicating the compound of Formula I and AMG 510 combine synergistically to inhibit cellular proliferation in KRAS G12C mutation in NCI-H358 cells.

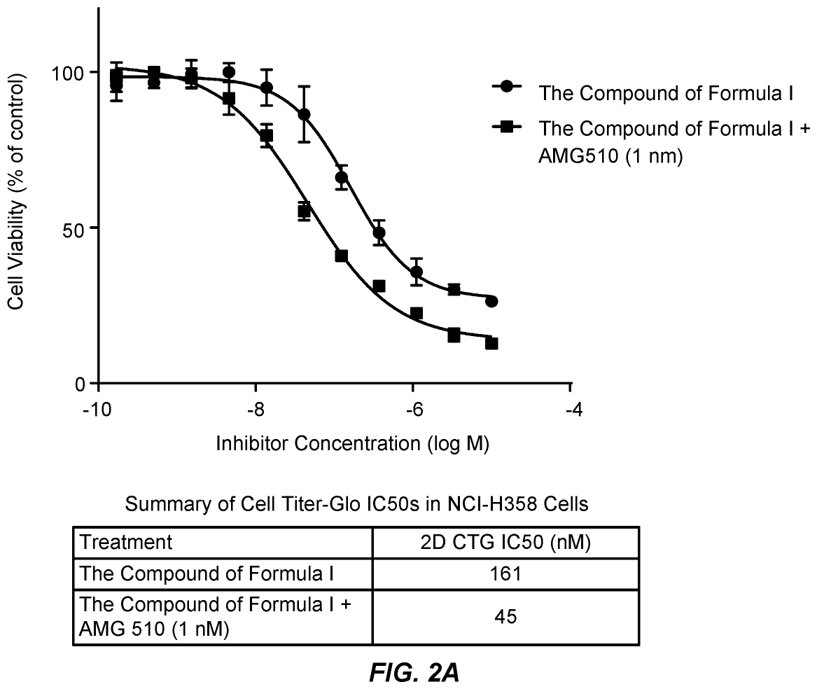

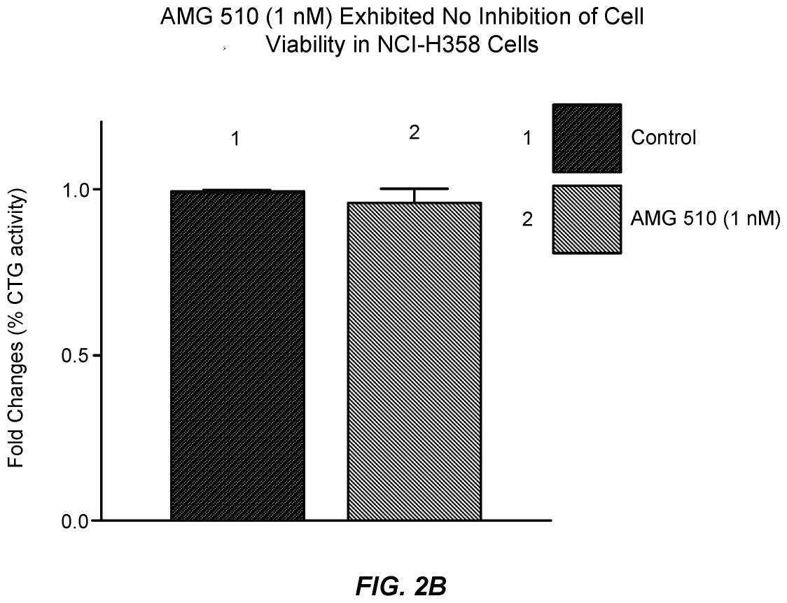

FIG. 2A shows a plot of percent activity versus inhibitor concentration (log M) in NCI-H358 cells treated with the compound of Formula I alone and in combination with AMG 510. Tabulated IC50 data in NCI-H358 cells treated with the compound of Formula I alone and in combination with AMG 510.

FIG. 2B shows a bar graph of percent CTG activity that indicates AMG 510 (1 nM) alone did not decrease cell viability in NCI-H358 cells.

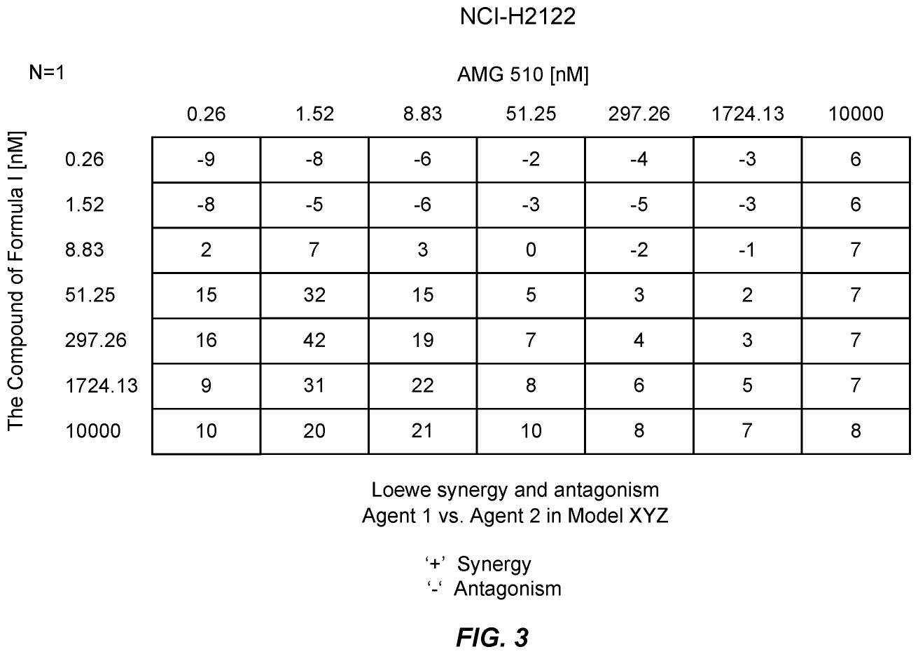

FIG. 3 shows data indicating the compound of Formula I and AMG 510 combine synergistically to inhibit cellular proliferation in KRAS G12C mutated NCI-H2122.

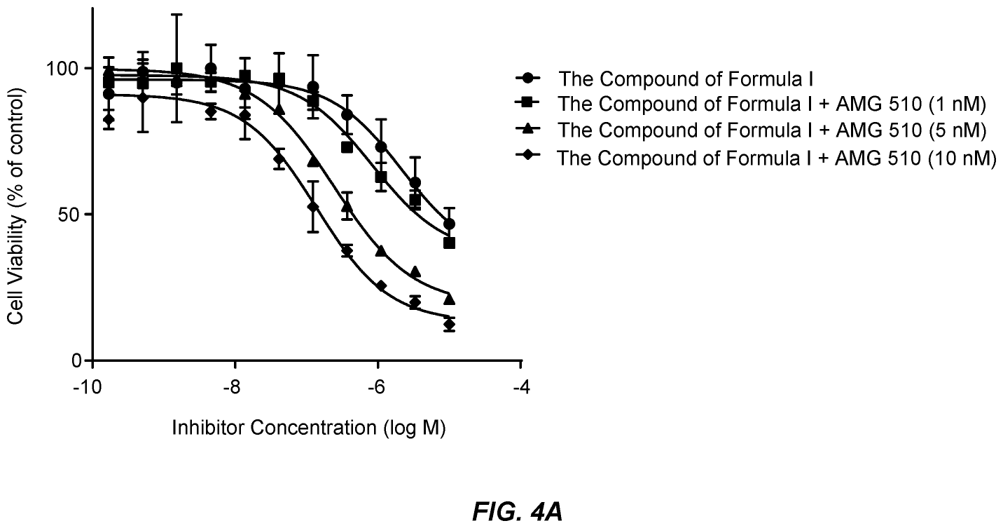

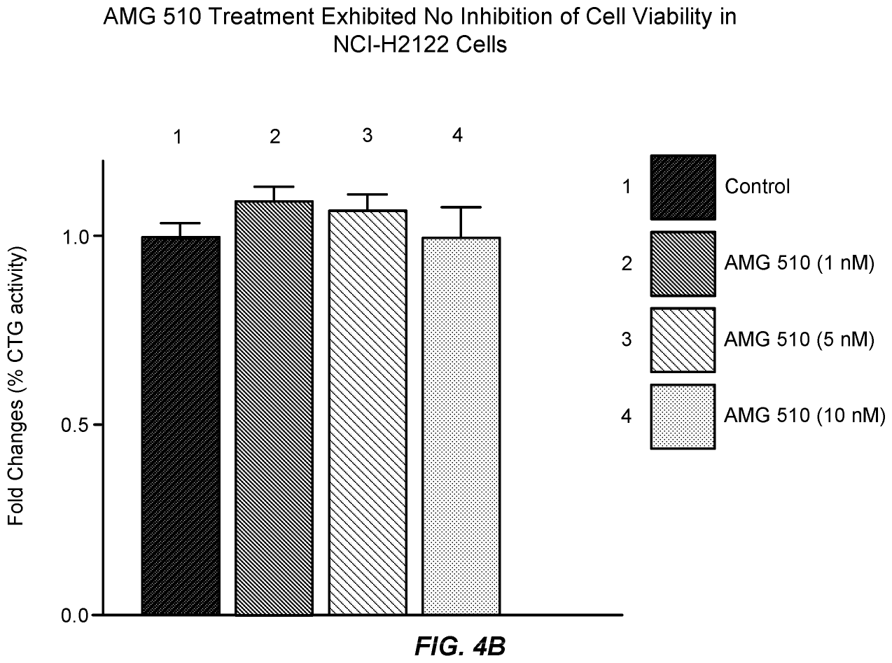

FIG. 4A shows a plot of percent activity versus inhibitor concentration (log M) in NCI-H2122 cells treated with the compound of Formula I alone and in combination with various concentrations of AMG 510.

FIG. 4B shows a bar graph of percent CTG activity that indicates AMG 510 (1 nM) alone did not decrease cell viability in NCI-H2122 cells.

Collectively, this data set indicates that the combination of the compound of Formula I and inhibitors of KRAS G12C provides synergistic inhibition of KRAS G12C mutated cancer cell viability. The activity of the compound of Formula I can be synergistically enhanced by combining with the inhibitors of KRAS G12C in cells bearing the KRAS G12C mutation.

### Example 2

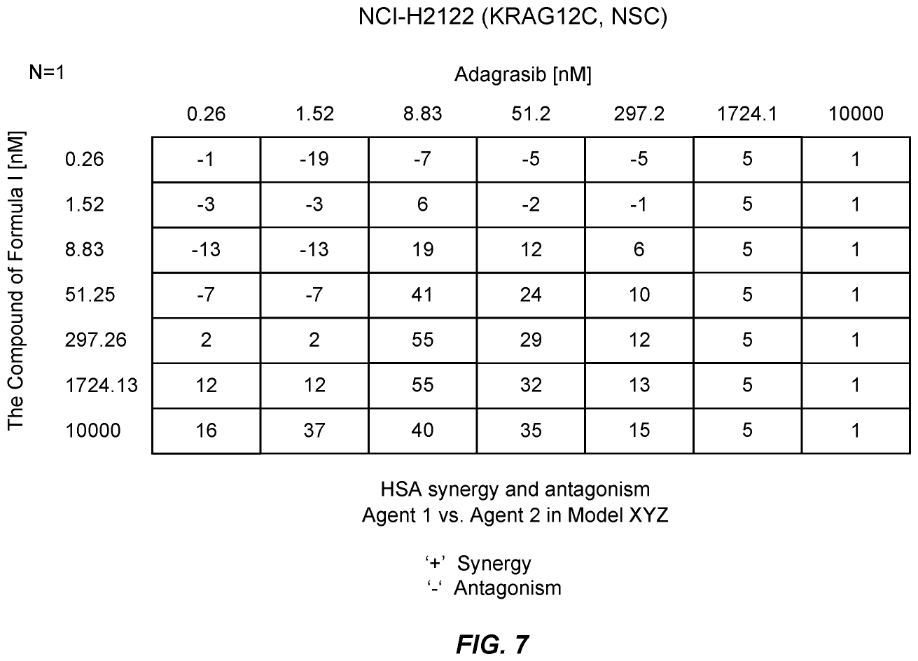

The results of these experiments are indicated in FIGS. 7 to 10, which are further discussed below.

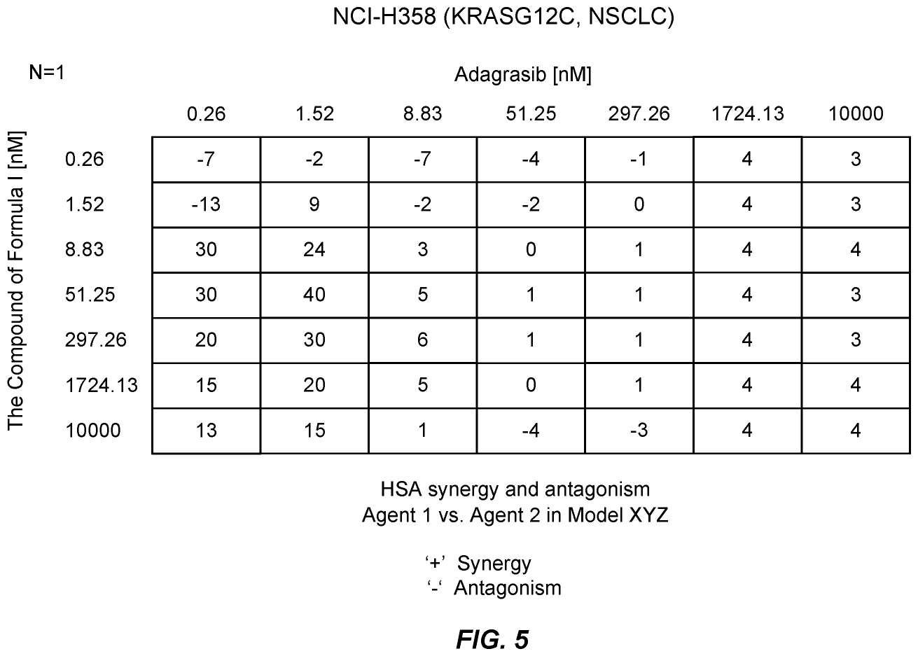

FIG. 5 shows a matrix representation of HSA synergy and antagonism, indicating the compound of Formula I and adagrasib combine synergistically to inhibit cellular proliferation in KRAS G12C mutated NCI-H358 cells.

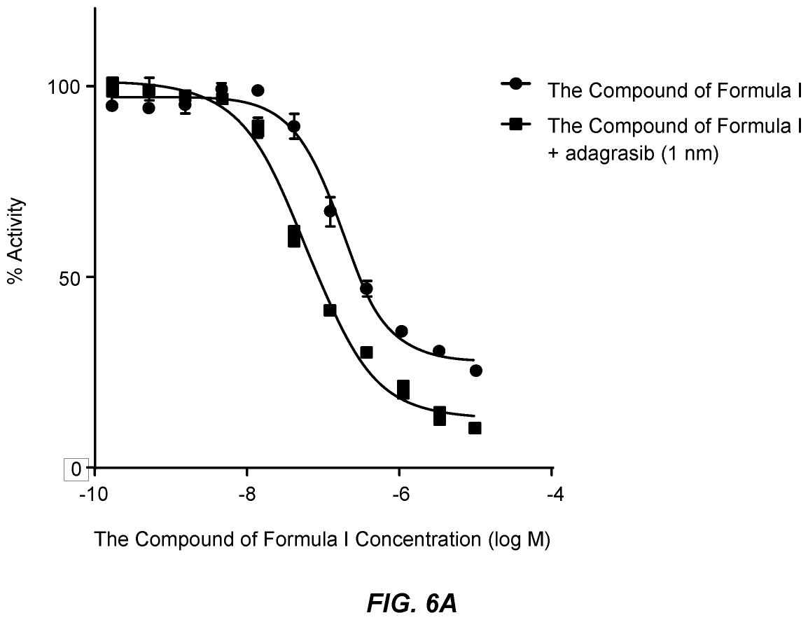

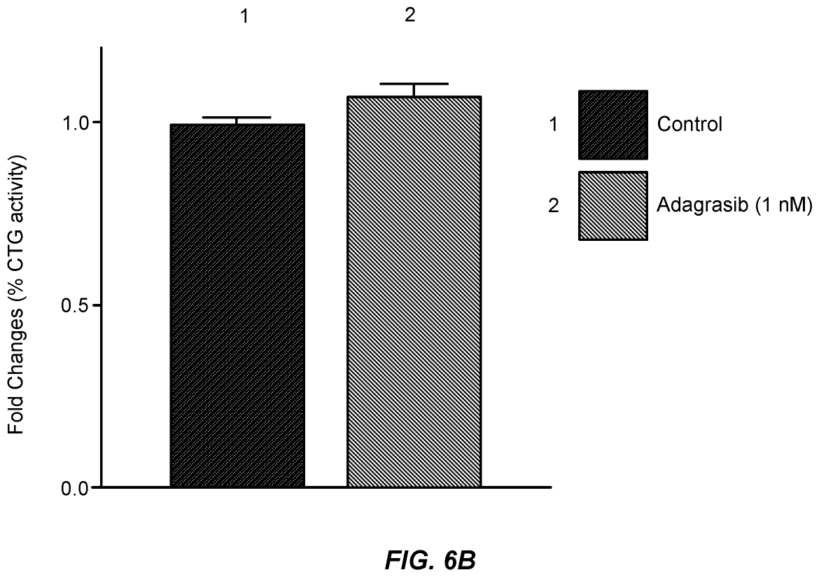

FIG. 6A shows a plot of percent activity versus inhibitor concentration (log M) in NCI-H358 cells treated with the compound of Formula I alone (solid circles) and in combination (solid squares) with 1 nM of adagrasib.

FIG. 6B shows a bar graph of percent CTG activity that indicates adagrasib alone at 1 nM did not decrease cell viability in NCI-H358 cells

Table 1. Summary of CellTiter-Glo IC50s in NCI-H358 Cells.

TABLE 1

| Treatment | 2D CTG IC50 (nM) |
|---|---|
| The compound of Formula I | 168 |
| The compound of Formula I + | 61 |
| adagrasib (1 nM) | |

FIG. 7 shows a matrix representation of HSA synergy and antagonism, indicating the compound of Formula I and adagrasib combine synergistically to inhibit cellular proliferation in KRAS G12C mutated NCI-H2122 cells.

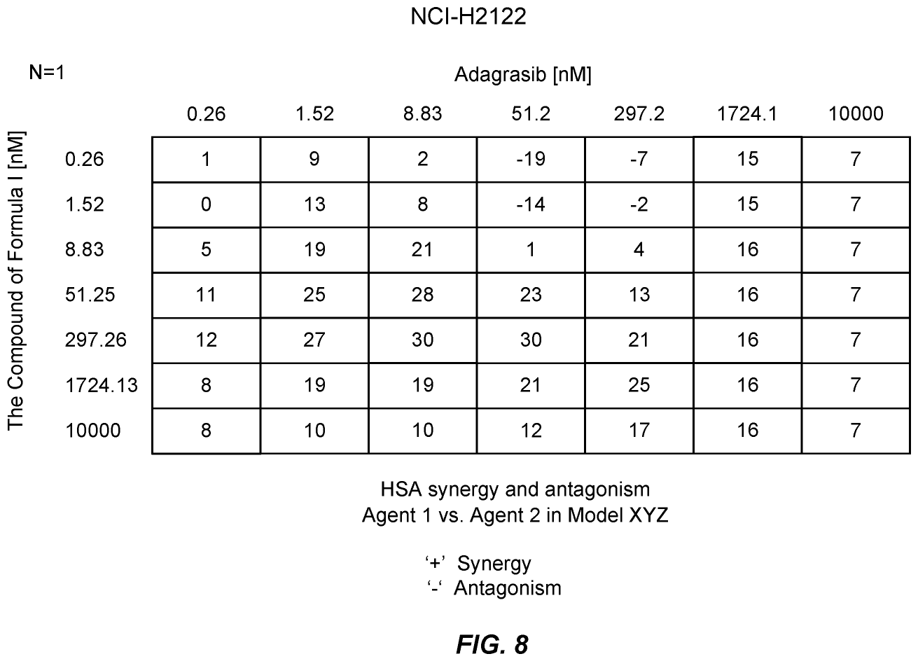

FIG. 8 shows a matrix representation of HSA synergy and antagonism, indicating the compound of Formula I and adagrasib combine synergistically to inhibit cellular proliferation in KRAS G12C mutated KYSE-410 cells.

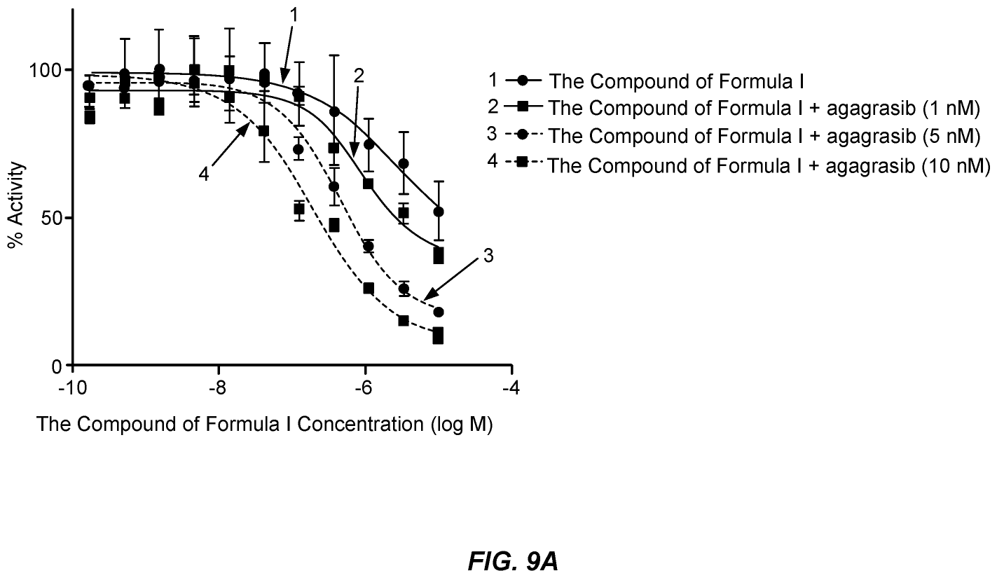

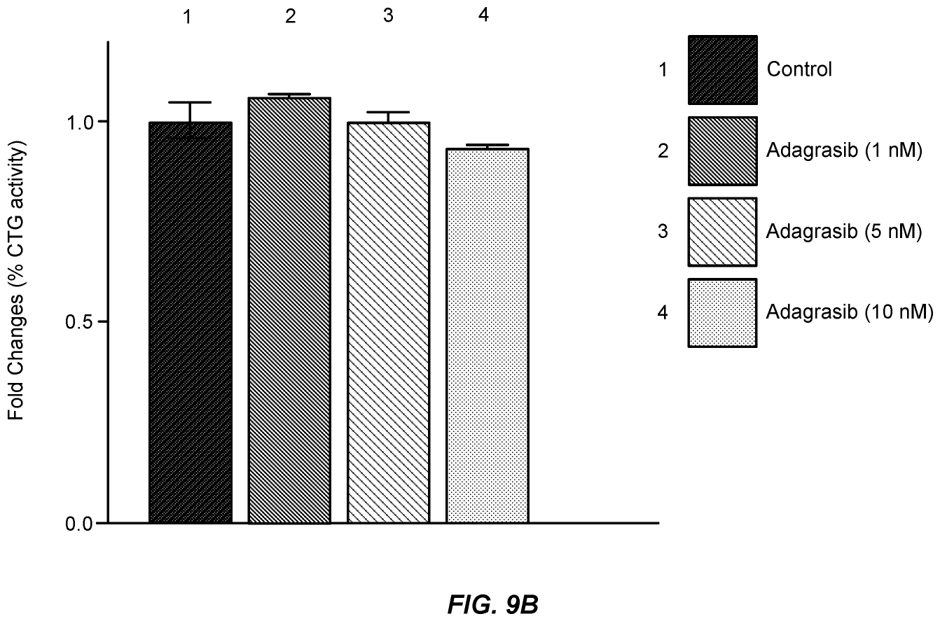

FIG. 9A shows a plot of percent activity versus inhibitor concentration (log M) in NCI-H2122 cells treated with the compound of Formula I alone (solid circles, Line 1) and in combination with 1 nM (solid squares, Line 2), 5 nM (solid circles, Line 3), or 10 nM (solid squares, Line 4) of adagrasib.

FIG. 9B shows a bar graph of percent CTG activity indicating that 1 nM, 5 nM or 10 nM of adagrasib alone did not decrease cell viability in NCI-H2122 cells.

Table 2. Summary of CellTiter-Glo IC50s in NCI-H2122 Cells.

TABLE 2

| Treatment | 2D CTG IC50 (nM) |
|---|---|
| The compound of Formula I | 2785 |
| The compound of Formula I + | 888 |
| adagrasib (1 nM) | |
| The compound of Formula I + | 460 |
| adagrasib (5 nM) | |
| The compound of Formula I + | 190 |
| adagrasib (10 nM) | |

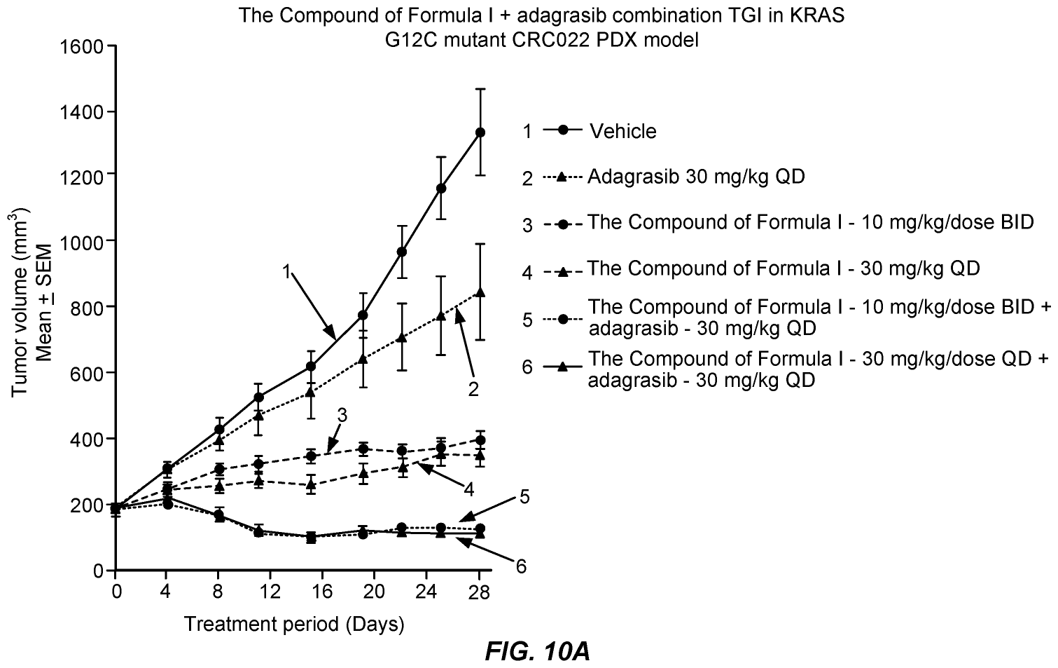

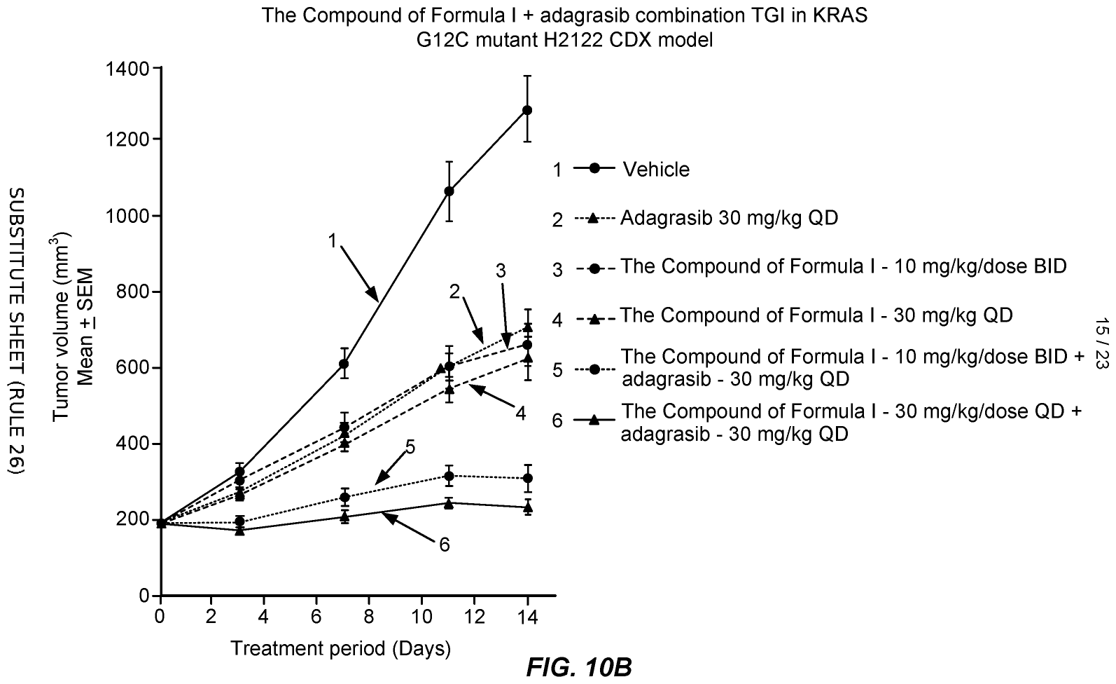

FIG. 10A shows a plot of tumor volume (mm³) versus treatment period (days) for a KRAS G12C mutated CRC022 PDX tumor xenograft model treated with vehicle (solid circles, Line 1), adagrasib alone (30 mg/kg QD, solid triangles, Line 2), the compound of Formula I alone at 10 mg/kg/dose BID (solid circles, Line 3), the compound of Formula I at 30 mg/kg QD dose (solid triangles, Line 4), the combination of the compound of Formula I (10 mg/kg/dose BID) and adagrasib at 30 mg/kg QD (solid circles, Line 5), and the combination of the compound of Formula I (30 mg/kg/QD) and adagrasib at 30 mg/kg QD (solid triangles, Line 6).

FIG. 10B shows a plot of tumor volume (mm³) versus treatment period (days) for a KRAS G12C mutated H2122 CDX tumor xenograft model treated with vehicle (solid circles, Line 1), adagrasib alone (30 mg/kg QD, solid triangles, Line 2), the compound of Formula I alone (10 mg/kg/dose BID, solid circles, Line 3), the compound of Formula I alone at 30 mg/kg QD (solid triangles, Line 4), the combination of the compound of Formula I (10 mg/kg/dose BID) and adagrasib at 30 mg/kg QD (solid circles, Line 5), and the combination of the compound of Formula I (30 mg/kg/QD) and adagrasib at 30 mg/kg QD (solid triangles, Line 6).

### Example 3: Combination of the Compound of Formula I and Adagrasib Showed Synergistic Effects on Cellular Proliferation in KRAS^{G12C} Cells

Cell lines were obtained from ATCC (NCI-H358 #CRL-5807 and NCI-H2122 #CRL-5985). KYSE-410 was obtained from Millipore Sigma (#94072023). The cells were cultured in RPMI with 10% of FBS and Pencillion/Stremtomycin and maintained at 37° C./5% CO₂.

Cellular proliferation assay: The cells (2000 cells per well) were plated onto 96-well plates in 100 μl cell culture medium. The cells were treated with the compound of Formula I and adagrasib with concentrations varying from 0 to 10 μM by using the Tecan D300e Digital Dispenser combination matrix protocol. At day 5, 50 μl of CellTiter-Glo (CTG) reagent (Promega) was added and the plates were incubated for 10 minutes with gentle shaking. After the 10 minutes incubation, the luminescent signal was determined according to the provider's instruction (Promega), and combination data was generated by Combenefit software.

Adagrasib sensitive cells, NCI-H358, were split onto 96 well plates. After overnight incubation, the compound of Formula I and adagrasib were added to the cells by using the Tecan D300e Digital Dispenser combination matrix protocol. A CellTiter-Glo assay was executed after 5 days of incubation and combination synergy was calculated by the standard HSA model using Combenefit software. The data were represented by area and intensity of color codes where synergy was represented by blue, additive was represented by green, and antagonism was represented by red. The combination data showed synergistic effects on cellular viability in NSCLC KRAS^{G12C} mutated cells NCI-H358 (FIG. 5).

The combination was studied by a cell viability assay. NCI-H358 cells were split onto a 96-well plate. After overnight incubation, the cells were treated with either the compound of Formula I alone or the combination of the compound of Formula I and a fixed final concentration of adagrasib (1 nM), and a CellTiter-Glo assay was executed after 5 days. Adagrasib (1 nM) treatment alone showed no inhibition of cell viability in NCI-H358 (FIG. 6A). However, a fixed concentration of adagrasib (1 nM) increased the compound of Formula I sensitivity in NCI-H358 cells (FIG. 6A). The IC50 of the compound of Formula I was reduced ˜3× with 1 nM final concentration of adagrasib treatment.

To maximize therapeutic response in KRAS^{G12C} cells that were moderately sensitive to KRAS^{G12C} inhibitors, we tested the combination of the compound of Formula I and adagrasib in NCI-H2122 and KYSE-410 cells. The cells were split onto 96 well plates. After overnight incubation, the compound of Formula I and adagrasib were added to the cells by using the Tecan D300e Digital Dispenser combination matrix protocol. A CellTiter-Glo assay was executed after 5 days of incubation and combination synergy was calculated by the standard HSA model using Combenefit software. The data were represented by area and intensity of color codes where synergy was represented by dark grey, additive was represented by light grey, and antagonism was represented by grey. The combination data showed synergistic effects on cellular viability in NCI-H2122 and KYSE-410 cells harboring KRAS^{G12C} mutation (FIG. 7 and FIG. 8).

The combination was confirmed by a follow-up viability assay. NCI-H2122 cells were split onto a 96-well plate. After overnight incubation, the cells were treated with either the compound of Formula I alone or the combination of the compound of Formula I and fixed final concentrations of adagrasib (1, 5, and 10 nM), and a CellTiter-Glo assay was executed after 5 days. Adagrasib treatment alone showed less than 10% inhibition of cell viability in NCI-H2122 (FIG. 9B). However, fixed concentration of adagrasib increased the compound of Formula I sensitivity dose dependently in NCI-H2122 cells (FIG. 9A). The IC50 of the compound of Formula I was reduced dose dependently from 2785 nM to 190 nM with adagrasib treatment. Overall, the data suggested that the combination of the compound of Formula I and adagrasib was effective in KRAS^{G12C} mutated human cancer cells.

### Example 4-A Phase 1b/2 Study of Agents Targeting the Mitogen-Activated Protein Kinase Pathway in Patients with Advanced Non-Small-Cell Lung Cancer

This study will include: 1) the evaluation of the safety and tolerability of escalating doses of the compound of Formula I in combination with other cancer therapies in study participants with advanced non-small cell lung cancer (NSCLC); 2) the determination of the Maximum Tolerated Dose (MTD) and/or Recommended Dose (RD) of the compound of Formula I administered in combination with other cancer therapies; 3) the evaluation of the antitumor activity of the compound of Formula I in combination with other cancer therapies; and 4) the evaluation of the pharmacokinetic (PK) profiles of the compound of Formula I and other cancer therapies when administered in combination.

The Phase 1b/2 study will include evaluating safety, tolerability, and antitumor activity of the compound of Formula I in combination with other cancer therapies in study participants with advanced NSCLC. The study will include a dose escalation cohort in which the compound of Formula I plus sotorasib is administered to study participants with advanced NSCLC harboring Kirsten rat sarcoma G12C mutation (KRAS G12Cm). The compound of Formula I will be orally administered in combination with sotorasib to study participants with KRAS G12Cm NSCLC in sequential ascending doses until unacceptable toxicity, disease progression, or withdrawal of consent. Dose expansion will follow and will evaluate the compound of Formula I orally administered at the RD identified from the respective dose escalation cohort in study participants with advanced EGFRm or KRAS G12Cm NSCLC.

### Inclusion Criteria

Age≥18 years.

Willing and able to give written informed consent.

Have histologically or cytologically confirmed NSCLC, with presence of EGFR mutation(s) sensitive to EGFR inhibitors, or KRAS G12C mutation.

Measurable disease per Response Evaluation Criteria in Solid Tumors (RECIST) v1.1.

Adequate bone marrow and organ function. Have Eastern Cooperative Oncology Group (ECOG) performance status of 0 or 1:
grade 0: fully active, able to carry on all pre-disease performance without restriction;
grade 1: restricted in physically strenuous activity but ambulatory and able to carry out work of a light or sedentary nature, e.g. light housework, office work.

Willing to comply with all protocol-required visits, assessments, and procedures.

Able to swallow oral medication.

### Exclusion Criteria

Concurrent treatment with any systemic anticancer therapy for NSCLC, including any approved or investigational agent.

For participants with KRAS G12Cm NSCLC: prior therapy with a SHP2, ERK, or KRAS G12C inhibitor (depending on which cohort is being considered for enrollment).

Palliative radiotherapy within 7 days of enrollment.

History of unacceptable toxicity to treatment with sotorasib.

Major surgery within the 28 days of enrollment.

Unresolved toxicities from prior systemic therapy greater than NCI Common Terminology Criteria for Adverse Events (CTCAE) grade 1 at time of enrollment, except for toxicities not considered a safety risk (e.g., alopecia, vitiligo, and grade 2 neuropathy due to prior chemotherapy).

History of another malignancy≤5 years prior to first dose, except for patients who are disease-free for >2 years after treatment with curative intent or who have carcinoma in situ.

Symptomatic and unstable brain metastases, or spinal cord compression, except for patients who have completed definitive therapy (surgery or radiotherapy), are not on steroids, and have a stable neurologic status for a least 2 weeks after completion of the definitive therapy and steroids.

History of or clinically active Interstitial Lung Disease (ILD), drug induced ILD, or radiation pneumonitis that required steroid treatment.

Impaired cardiovascular function or clinically significant cardiovascular disease.

History or current evidence of retinal pigment epithelial detachment (RPED), central serous retinopathy, retinal vein occlusion (RVO), or predisposing factors to RPED or RVO.

Any evidence of severe or uncontrolled systemic disease or evidence of any other significant clinical disorder or laboratory finding that renders the patient inappropriate to participate in the study.

Pregnant or breastfeeding women.

Contraindication to sotorasib use as per local label.

### Example 5—In Vivo Studies of the Compound of Formula I Alone, Sotorasib Alone, and the Compound of Formula 1+Sotorasib Combination in a KRAS G12C Mutant NSCLC CDX Model, SW1573

### Materials and Methods

The vehicle/control article, 100 mM acetic acid in deionized water, with pH adjustment to 4.8-5.0, was prepared and stored under ambient conditions throughout the 27-day administration in mice. The test article of the compound of Formula 1 was prepared in vehicle of 100 mM acetic buffer weekly and stored under ambient conditions. The combination agent sotorasib was prepared in vehicle of 50%/50% w/w PEG400/PG, acidified by HCl weekly and stored at 2-8° C.

Female Balb/c nude mice were purchased from Vital River (Beijing, China). Mice were between 6-8 weeks of age at the time of implantation. Mice were hosted at animal rooms of a vivarium facility and acclimated to their new environment for at least 3 days prior to initiation of any experiments. All procedures related to animal handling, care and treatment in this study were performed according to guidelines approved by the Institutional Animal Care and Use Committee (IACUC) of GenenDesign (Shanghai, China). In addition, all portions of this study were performed at GenenDesign and adhered to the study protocol approved by the study director and applicable standard operating procedures (SOPs).

### Preparation of CDX

SW1573 was a human NSCLC cell line that harbored a KRAS G12C mutation. The cell line was purchased from ATCC. Early passage SW1573 cells were maintained in vitro as a monolayer culture in L-15 medium supplemented with 10% fetal bovine serum (FBS) at 37° C. in an atmosphere of 5% CO₂ in air. The medium was renewed every 2 to 3 days and tumor cells were routinely sub-cultured at a confluence of 80-90% by trypsin-EDTA and did not exceed 5 passages. The cells growing in an exponential growth phase were harvested and counted for tumor inoculation into mice.

Mice were anesthetized by isoflurane before subcutaneous implantation. 200 μL cell suspensions containing 5×10⁶ SW1573 tumor cells mixed with 50% Matrigel were implanted into the right flank of the mouse subcutaneously using a syringe. Animal health and tumor growth were monitored daily after implantation. Tumor volume was measured twice a week by caliper when xenograft tumors were palpable and measurable. When subcutaneous tumor volumes reached a mean of approximately 200 mm³ (range of 150-263 mm³), tumor-bearing mice were randomized into different groups (n=8 mice per group). The randomization date was denoted as treatment day 0.

### Treatment

Tumor-bearing mice were treated on the day of randomization. The treatment start day was denoted as treatment day 0. Mice were dosed by oral administration of vehicle control, the compound of Formula I monotherapy at 10 mg/kg/dose BID and 30 mg/kg QD, and sotorasib monotherapy at 30 and 100 mg/kg QD. Mice were also treated in two combination treatment groups of the compound of Formula I+sotorasib, with one group dosed with the combination of the compound of Formula I at 10 mg/kg/dose BID and sotorasib at 100 mg/kg QD, and the other group dosed with the combination of the compound of Formula I at 30 mg/kg QD with sotorasib at 100 mg/kg QD. The dosing volume was 5 mL/kg, and the interval of the BID regimen was 8 hours. The study was terminated when the criteria of termination defined in the study protocol were met.

### Results

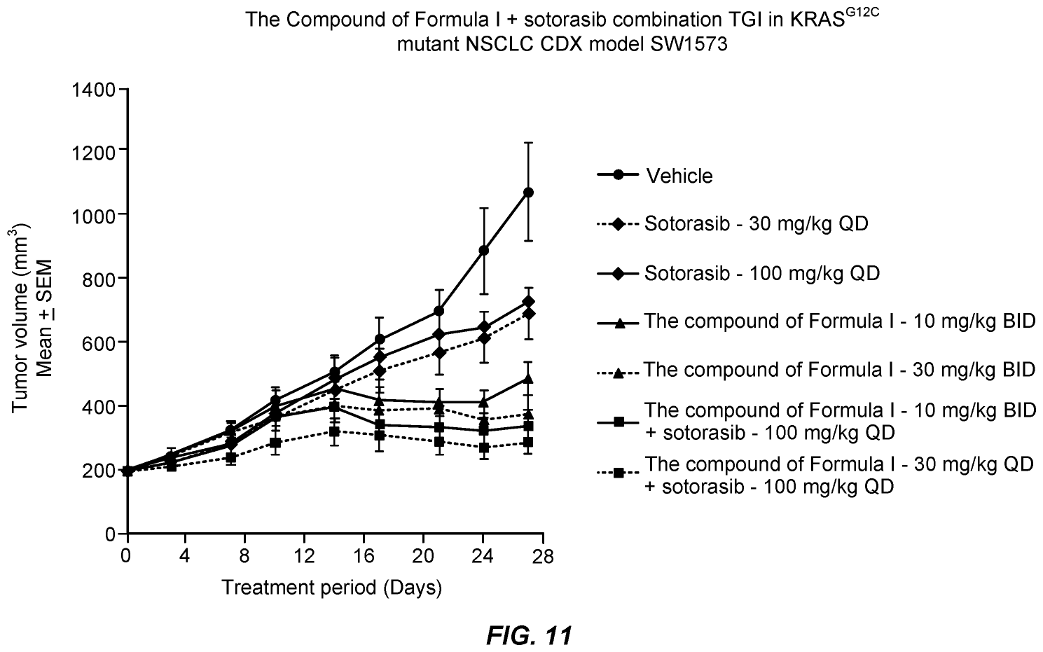

FIG. 11 shows a graph of tumor volume over a period of treatment time with a regimen of the compound of Formula I alone, sotorasib alone, and the combination of the compound of Formula I and sotorasib in KRAS G12C mutant NSCLC CDX model SW1573. No significant body weight change was observed in the control and treatment groups.

### Conclusion

As illustrated by FIG. 11, the combination of the compound of Formula I and sotorasib demonstrated superior tumor growth inhibition relative to the respective monotherapies in the KRAS G12C mutant NSCLC CDX model SW1573.

### Example 6: In Vivo Studies of the Compound of Formula I Alone, Sotorasib Alone, and the Compound of Formula I+Sotorasib Combination in a KRAS G12C Mutant NSCLC CDX Model, NCI-H358

### Materials and Methods

The vehicle/control article, 100 mM acetic acid in deionized water, with pH adjustment to 4.8-5.0, was prepared and stored under ambient conditions throughout the 28-day administration in mice. The test article of the compound of Formula I was prepared in vehicle of 100 mM acetic buffer throughout the 28-day administration and stored under ambient conditions. The combination agent sotorasib was prepared in vehicle of 50% PEG 400/50% PG, acidified by HCl throughout the 28-day administration and stored at 2-8° C.

Female nude (Nu/nu) mice were purchased from Jackson Laboratory (US). Mice were between 6-8 weeks of age at the time of implantation. Mice were hosted at animal rooms of a vivarium facility and acclimated to their new environment for 3 days prior to initiation of any experiments.

### Preparation of CDX

NCI-H358 was a human NSCLC cell line that harbored a KRAS G12C mutation. The cell line was purchased from ATCC. Early passage NCI-H358 cells were maintained in vitro as monolayer culture in RPMI 1640 medium supplemented with 10% fetal bovine serum (FBS) at 37° C. in an atmosphere of 5% CO₂ in air. The medium was renewed every 2 to 3 days and tumor cells were routinely sub-cultured at a confluence of 80-90% by trypsin-EDTA, and did not exceed 5 passages. The cells growing in an exponential growth phase were harvested and counted for tumor inoculation into mice.

Mice were anesthetized by isoflurane before subcutaneous implantation. 200 μL cell suspensions containing 5×10⁶ NCI-H358 tumor cells mixed with 50% Matrigel were implanted into the right flank of the mouse subcutaneously using a syringe. Animal health and tumor growth were monitored daily after implantation. Tumor volume was measured twice a week by caliper when xenograft tumors were palpable and measurable. When subcutaneous tumor volumes reached a mean of approximately 300 mm³ (range of 150-390 mm³), tumor-bearing mice were randomized into different groups (n=10 mice per group). The randomization date was denoted as treatment day 0.

### Treatment

Treatment started the day after randomization. The treatment start date was denoted as treatment day 1. Mice were dosed by oral administration of vehicle, the compound of Formula I monotherapy at 10 mg/kg/dose BID and 30 mg/kg QD, and sotorasib monotherapy at 10 mg/kg QD. Mice were also treated in two combination treatment groups of the compound of Formula I+sotorasib, with one group dosed with the compound of Formula I at 10 mg/kg/dose BID and sotorasib at 10 mg/kg QD, and the other group dosed with the compound of Formula I at 30 mg/kg QD and sotorasib at 10 mg/kg QD. The dosing volume was 5 mL/kg and the interval of the BID regimen was 8 hours. The compound of Formula I was dosed first, followed by sotorasib an hour later in the combination treatment groups. The study was terminated when the criteria of termination defined in the study protocol were met.

### Results

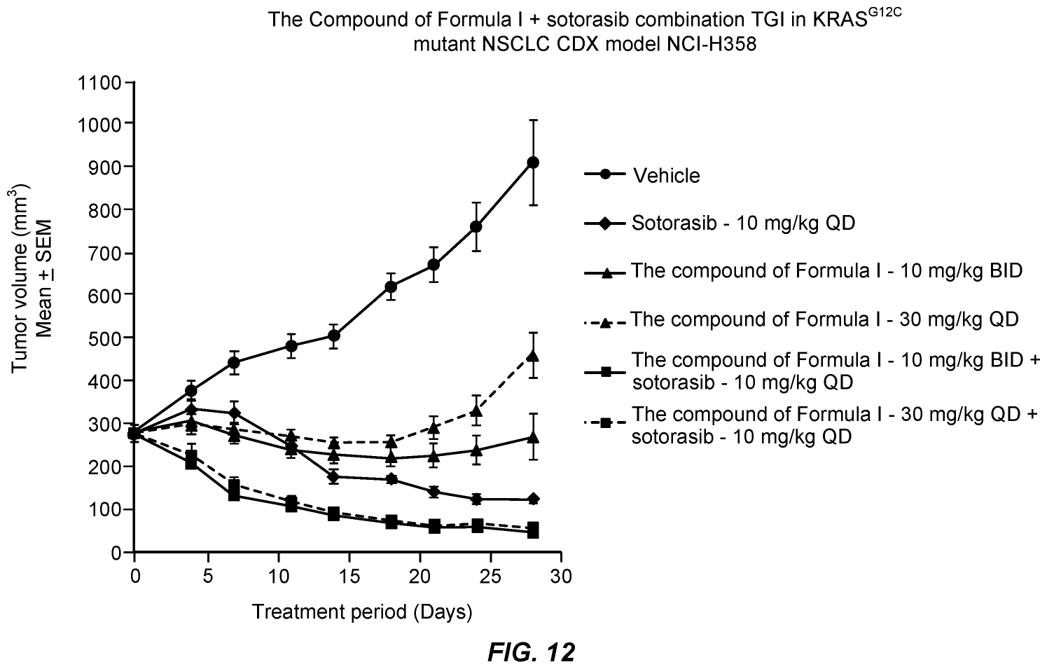

FIG. 12 shows a graph of tumor volume over a period of treatment time with a regimen of the compound of Formula I alone, sotorasib alone, and the combination of the compound of Formula I and sotorasib in KRAS G12C mutant NSCLC CDX model NCI-H358. No significant body weight change was observed in the control and treatment groups.

### Conclusion

As illustrated by FIG. 12, the combination of the compound of Formula I and sotorasib demonstrated superior tumor growth inhibition relative to the respective monotherapies in the KRAS G12C mutant NSCLC CDX model NCI-H358.

### Example 7: In Vivo Studies of the Compound of Formula I Alone, Sotorasib Alone, and Formula 1+Sotorasib Combination in a KRAS G12C Mutant Esophageal Squamous Cell Carcinoma CDX Model, KYSE-410

### Materials and Methods

The vehicle/control article, 100 mM acetic acid in deionized water, with pH adjustment to 4.8-5.0, was prepared and stored under ambient conditions throughout the 21-day administration in mice. The test article of the Compound of Formula I was prepared in vehicle of 100 mM acetic buffer weekly and stored under ambient conditions. The combination agent sotorasib was prepared in vehicle of 50% w/w polyethylene glycol 400 (PEG400)+50% w/w propylene glycol (PG) and stored at 2-8° C.

Female nude (Nu/nu) mice were purchased from Jackson Laboratory (US). Mice were between 6-7 weeks of age at the time of implantation. Mice were hosted at animal rooms of a vivarium facility and acclimated to their new environment for at least 3 days prior to initiation of any experiments. All procedures related to animal handling, care, and treatment in this study were performed according to guidelines approved by the Institutional Animal Care and Use Committee (IACUC) of Explora BioLabs (San Diego, CA). In addition, all portions of this study performed at Explora BioLabs adhered to the study protocols approved by the study director and applicable standard operating procedures (SOPs).

### Preparation of CDX

KYSE-410, a human esophageal squamous cell carcinoma cell line harboring a KRAS G12C mutation, was purchased from ATCC and was cultured in medium containing RPMI-1640 plus 10% fetal bovine serum (FBS) at 37° C. in an atmosphere of 5% CO₂ in air. The medium was renewed every 2 to 3 days, and tumor cells were routinely sub-cultured at a confluence of 80-90% by trypsin-EDTA. The cells growing in an exponential growth phase were harvested and counted for inoculation.

Mice were anesthetized by isoflurane before subcutaneous implantation. 200 μL cell suspensions containing 4×10⁶ KYSE-410 tumor cells mixed with 50% Matrigel were implanted into the right flank of the mouse subcutaneously using a syringe. Animal health and tumor growth were monitored daily after implantation. Tumor volume was measured twice a week by caliper when xenograft tumors were palpable and measurable. When subcutaneous tumor volumes reached a mean of approximately 196 mm³ (range of 150-300 mm³), tumor-bearing mice were randomized into different groups (n=10 mice per group). The randomization day was denoted as treatment day 0.

### Treatment

Treatment started on the day after randomization. The treatment start day was denoted as treatment day 1. Mice were dosed by oral administration of vehicle control solution, the Compound of Formula I alone at 10 mg/kg/dose BID, the Compound of Formula I at 30 mg/kg QD, or sotorasib at 100 mg/kg QD. Two additional groups received combination treatment of the Compound of Formula I and sotorasib, with one group dosed with the Compound of Formula I at 10 mg/kg/dose BID, and the other group dosed with the Compound of Formula I at 30 mg/kg QD; both combination groups were dosed with sotorasib at 100 mg/kg QD. The dosing volume was 5 mL/kg and the interval of the BID regimen was 8 hours. Sotorasib was dosed one hour after the first dose of the Compound of Formula I BID dose or QD dose in combination groups. The study was terminated on treatment day 21 as defined in the study protocol.

### Results

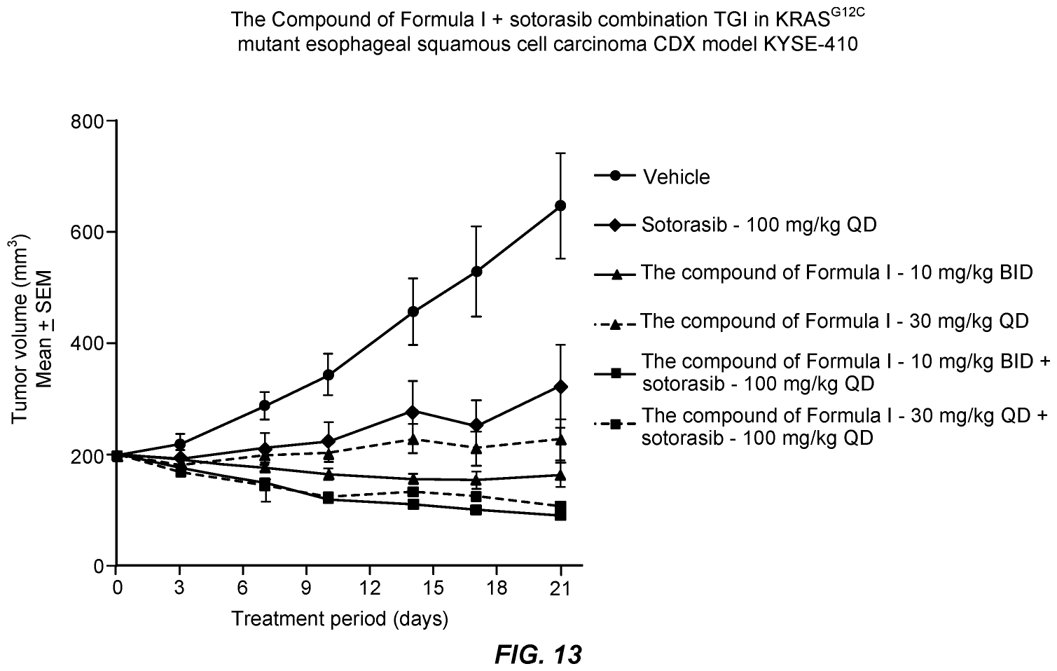

FIG. 13 shows a graph of tumor volume over a period of treatment time with a regimen of the compound of Formula I alone, sotorasib alone, and the combination of the compound of Formula I and sotorasib in KRAS G12C mutant esophageal squamous cell carcinoma CDX model KYSE-410. No significant body weight change was observed in the control and treatment groups.

### Conclusion

As illustrated by FIG. 13, the combination of the Compound of Formula I and sotorasib demonstrated superior tumor growth inhibition relative to the respective monotherapies in the KRAS G12C mutant esophageal squamous cell carcinoma CDX model KYSE-410.

### Example 8: In Vivo Studies of the Compound of Formula I Alone, Sotorasib Alone, and the Compound of Formula I+Sotorasib Combination in a KRAS G12C Mutant CRC PDX Model, CO-04-0310

### Materials and Methods

The vehicle/control article, 100 mM acetic acid in deionized water, with pH adjustment to 4.8-5.0, was prepared and stored under ambient conditions throughout the 28-day administration in mice. The test article of the Compound of Formula I was prepared in vehicle of 100 mM acetic buffer weekly and stored under ambient conditions. The combination agent sotorasib was prepared in vehicle of 50% w/w polyethylene glycol 400 (PEG400)+50% w/w propylene glycol (PG) and stored at 2-8° C.

Female Balb/c nude mice were purchased from the Beijing Vital River Laboratory Animal Technology Co., Ltd. Mice were between 6-8 weeks of age at the time of implantation. Mice were hosted in a special pathogen-free (SPF) environment of a vivarium facility and acclimated to their new environment for at least 3 days prior to initiation of any experiments. All procedures related to animal handling, care, and treatment in this study were performed according to guidelines approved by the Institutional Animal Care and Use Committee (IACUC) of WuXi AppTec. During the study, the care and use of animals were conducted in accordance with the regulations of the Association for Assessment and Accreditation of Laboratory Animal Care (AAALAC). In addition, all portions of this study performed at WuXi App Tec adhered to the study protocols approved by the study director and applicable standard operating procedures (SOPs).

### Preparation of PDX

The CO-04-0310 PDX model was established for preclinical efficacy study at WuXi AppTec. This PDX model was derived from an 82-year-old female Chinese CRC patient. A KRAS G12C mutation in the PDX model CO-04-0310 was confirmed by whole exome sequencing and PCR sequencing. Mouse skin was cleaned with appropriate surgical scrub and alcohol over the right flank. Tumor fragments (15-30 mm³) harvested from the PDX model were implanted subcutaneously in the right flanks of female Balb/c nude mice using a 18 g trochar needle. When tumor sizes reached 100-218 mm³ in volume, tumor-bearing mice were randomly divided into study groups with 8 mice in each group. The randomization date was denoted as treatment day 0.

### Treatment

Treatment started on the day after randomization. The treatment start day was denoted as treatment day 1. Mice were dosed by oral administration of vehicle control solution, the Compound of Formula I alone at 10 mg/kg/dose BID, the Compound of Formula I alone at 30 mg/kg QD, and sotorasib at 30 mg/kg QD. Two additional groups received combination treatment of the Compound of Formula I and sotorasib, with one group dosed with the Compound of Formula I at 10 mg/kg/dose BID and sotorasib at 30 mg/kg QD and the other group dosed with the Compound of Formula I at 30 mg/kg QD with sotorasib at 30 mg/kg QD. The dosing volume was 5 mL/kg and interval of BID regimen was 8 hours. The study was terminated on treatment day 28 as defined in the study protocol

### Results

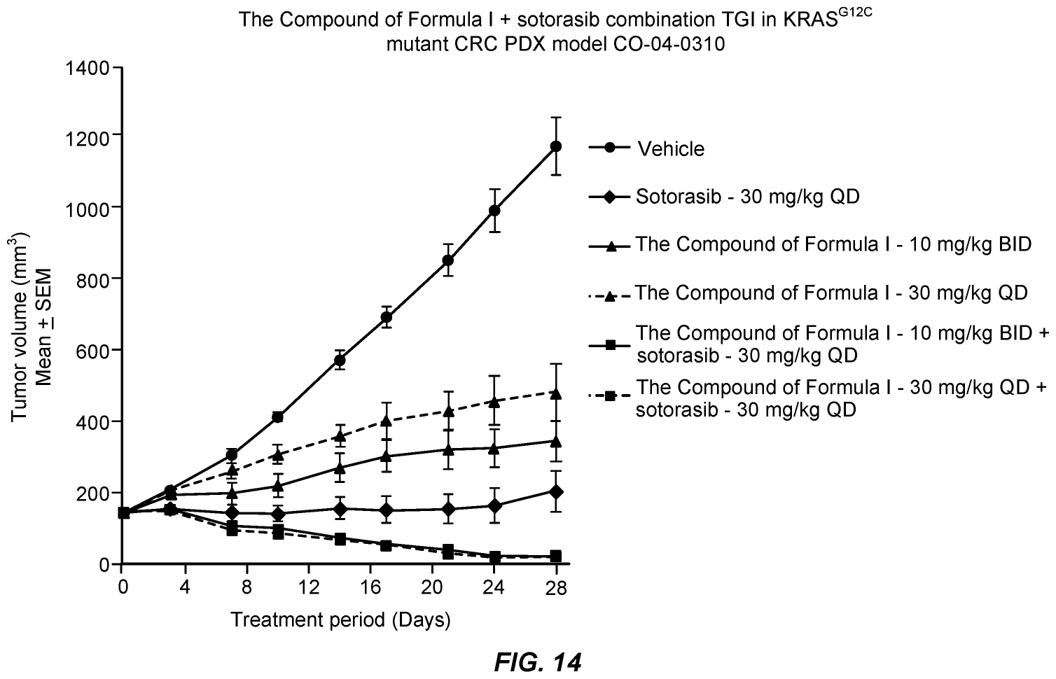

FIG. 14 shows a graph of tumor volume over a period of treatment time with a regimen of the compound of Formula I alone, sotorasib alone, and the combination of the compound of Formula I and sotorasib in KRAS G12C mutant CRC PDX model CO-04-0310. No significant body weight change was observed in the control and treatment groups.

### Conclusion

As illustrated by FIG. 14, the combination of the Compound of Formula I and sotorasib demonstrated superior tumor growth inhibition relative to the respective monotherapies in the KRAS G12C mutant CRC PDX model CO-04-0310.

### Example 9: In Vivo Studies of the Compound of Formula I Alone, Sotorasib Alone, and the Compound of Formula I+Sotorasib Combination in a KRAS G12C Mutant CRC PDX Model, CR2528

### Materials and Methods

The vehicle/control article, 100 mM acetic acid in deionized water, with pH adjustment to 4.8-5.0, was prepared and stored under ambient conditions throughout the 24-day administration in mice. The test article of the Compound of Formula I was prepared in vehicle of 100 mM acetic buffer weekly and stored under ambient conditions. The combination agent sotorasib was prepared in vehicle of 50% w/w polyethylene glycol 400 (PEG400)+50% w/w propylene glycol (PG) and stored at 2-8° C.

Female Balb/c nude mice were purchased from the SPF (Beijing) Laboratory Animal Technology Co, Ltd. (Beijing, China). Mice were between 7-9 weeks of age at the time of implantation. Mice were hosted in a special pathogen-free (SPF) environment of a vivarium facility and acclimated to their new environment for at least 3 days prior to initiation of any experiments. All procedures related to animal handling, care, and treatment in this study were performed according to guidelines approved by the Institutional Animal Care and Use Committee (IACUC) of Crown Bioscience (Beijing, China). During the study, the care and use of animals were conducted in accordance with the regulations of the Association for Assessment and Accreditation of Laboratory Animal Care (AAALAC). In addition, all portions of this study performed at Crown Bioscience (Beijing, China) adhered to the study protocols approved by the study director and applicable standard operating procedures (SOPs).

### Preparation of PDX

The CR2528 PDX model was established for preclinical efficacy study at CrownBio. This PDX model was derived from a 73-year-old male Chinese CRC patient. A KRAS G12C mutation in the PDX model CR2528 was confirmed by both RNA sequencing and exome sequencing. Mouse skin was cleaned with appropriate surgical scrub and iodophor over the right flank. Tumor fragments (2-3 mm in diameter) harvested from the PDX model were implanted subcutaneously in the right flanks of female Balb/c nude mice using a 18 g trochar needle. When mean tumor sizes reached 204 mm³ (range of 149-275 mm³), tumor-bearing mice were randomly divided into 6 study groups with 8 mice in each group. The randomization date was denoted as treatment day 0.

### Treatment

Treatment started on the day of randomization. The treatment start day was denoted as treatment day 0. Mice were dosed by oral administration of vehicle control solution, sotorasib at 30 mg/kg QD, the Compound of Formula I alone at 10 mg/kg/dose BID, and the Compound of Formula I alone at 30 mg/kg QD. Two additional groups received combination treatment of the Compound of Formula I and sotorasib, with one group dosed with the Compound of Formula I at 10 mg/kg/dose BID and sotorasib at 30 mg/kg QD, and the other group dosed with the Compound of Formula I at 30 mg/kg QD and sotorasib at 30 mg/kg QD. The dosing volume was 5 mL/kg and interval of BID regimen was 8 hours. Sotorasib was dosed one hour after the Compound of Formula I QD dose or the first BID dose in the combination groups.

### Results

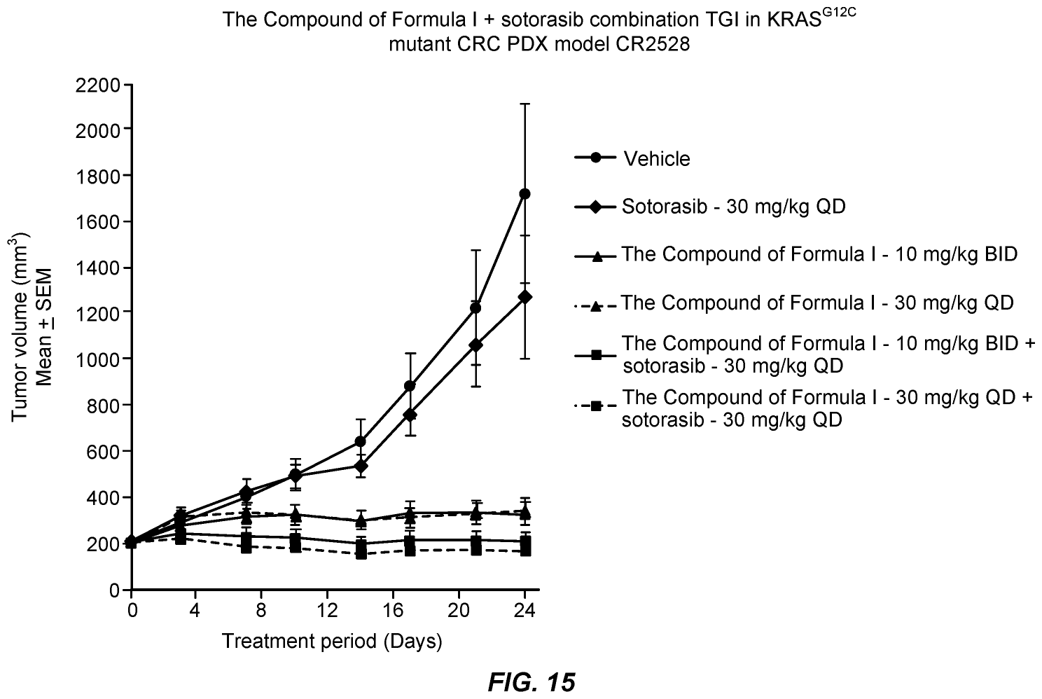

FIG. 15 shows a graph of tumor volume over a period of treatment time with a regimen of the compound of Formula I alone, sotorasib alone, and the combination of the compound of Formula I and sotorasib in KRAS G12C mutant CRC PDX model CR2528. No significant body weight change was observed in the control and treatment groups.

### Conclusion

As illustrated by FIG. 15, the combination of the Compound of Formula I and sotorasib demonstrated superior tumor growth inhibition relative to the respective monotherapies in the KRAS G12C mutant CRC PDX model CR2528.

### Example 10: In Vivo Studies of the Compound of Formula I Alone, Sotorasib Alone, and the Compound of Formula I+Sotorasib Combination in a KRAS G12C Mutant NSCLC CDX Model NCI-H2122

### Materials and Methods

The vehicle/control article, 100 mM acetic acid in deionized water, with pH adjustment to 4.8-5.0, was prepared and stored under ambient conditions throughout the 14-day administration in mice. The test article of the Compound of Formula I was prepared in vehicle of 100 mM acetic buffer weekly and stored under ambient conditions. The combination agent sotorasib was prepared in vehicle of 50% w/w polyethylene glycol 400 (PEG400)+50% w/w propylene glycol (PG) and stored at 2-8° C.

Female Balb/c nude mice were purchased from the Beijing Vital River Laboratory Animal Technology Co., Ltd. Mice were hosted in a special pathogen-free (SPF) environment of a vivarium facility and acclimated to their new environment for at least 3 days prior to the initiation of any experiments. Mice were between 6-8 weeks of age at the time of implantation. All procedures related to animal handling, care, and treatment in this study were performed according to the protocols and guidelines approved by the Institution al Animal Care and Use Committee (IACUC) of GenenDesign. Animal facility and program is operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

### Preparation of CDX

NCI-H2122, a human NSCLC cell line harboring a KRAS^{G12C} mutation, was purchased from ATCC and cultured in medium containing RPMI-1640 plus 10% fetal bovine serum (FBS) at 37° C. in an atmosphere of 5% CO₂ in air. The medium was renewed every 2 to 3 days and tumor cells were routinely sub-cultured at a confluence of 80-90% by trypsin-EDTA. The cells growing in an exponential growth phase were harvested and counted for inoculation. Mice were anesthetized by isoflurane before subcutaneous implantation. 200 μL cell suspensions containing 2×10⁶ NCI-H2122 tumor cells mixed with 50% Matrigel were implanted into the right flank of the mouse subcutaneously using a syringe. Animal health and tumor growth were monitored daily after implantation. Tumor volume was measured twice a week by caliper when xenograft tumors were palpable and measurable. When subcutaneous tumor volumes reached a mean of approximately 190 mm³ (range of 146-262 mm³), tumor-bearing mice were randomized into study groups (n=8 mice per group). The randomization day was denoted as treatment day 0.

### Treatment

Treatment started on the day of randomization. The treatment start day was denoted as treatment day 0. Mice were dosed by oral administration of vehicle control solution or monotherapy treatments of sotorasib at 100 mg/kg QD, or the Compound of Formula I at 30 mg/kg QD. One additional group received the combination treatment of the Compound of Formula I at 30 mg/kg QD and sotorasib at 100 mg/kg QD.

### Results

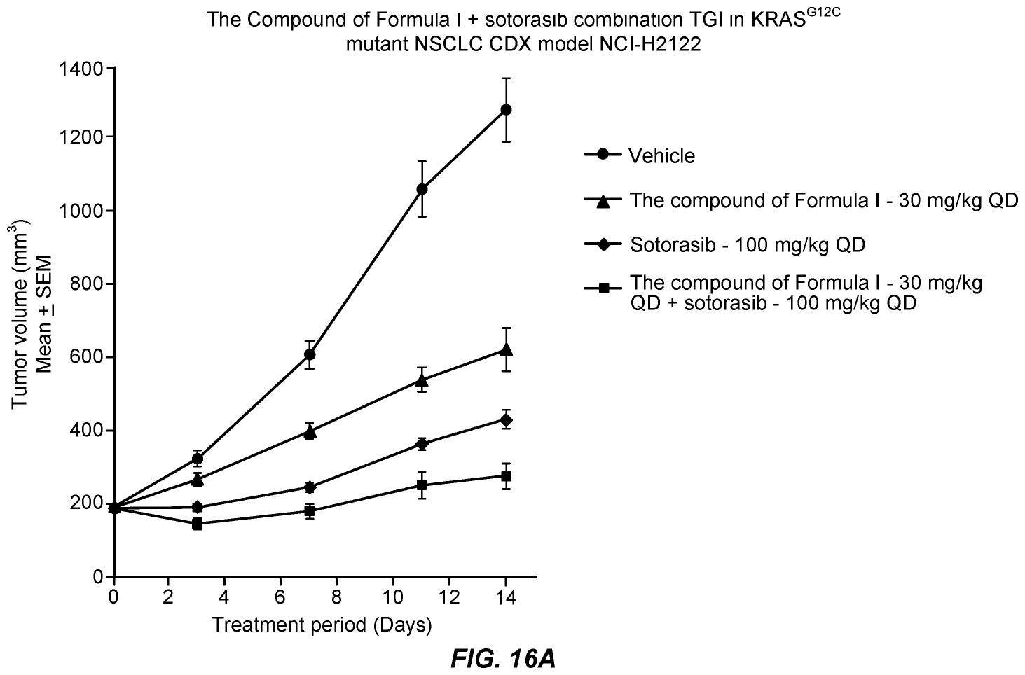

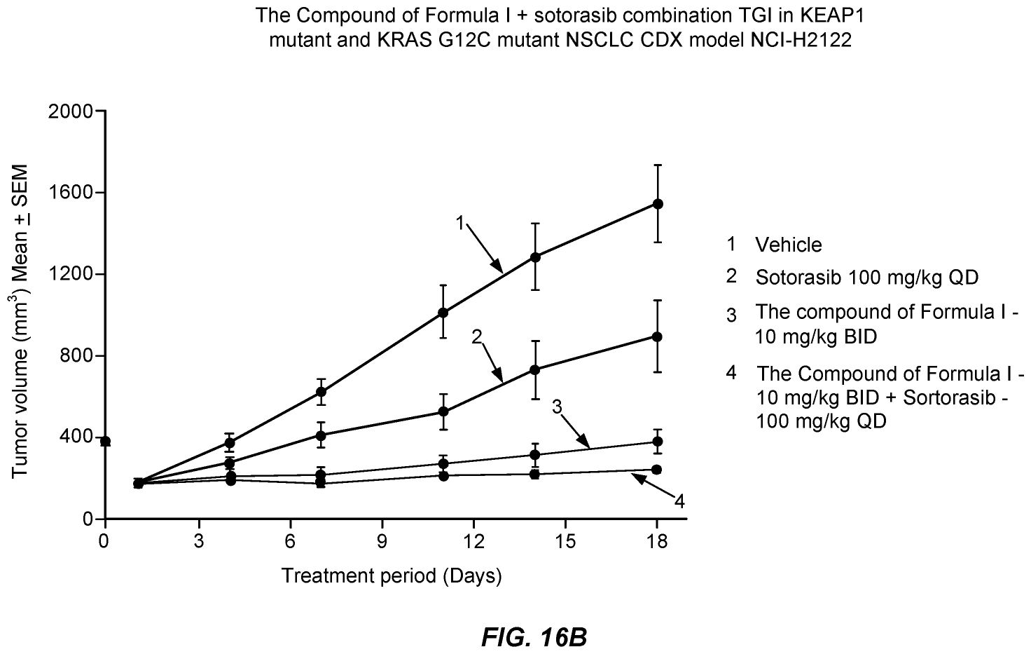

FIG. 16A shows a graph of tumor volume over a period of treatment time with a regimen of the compound of Formula I alone (30 mg/kg QD), sotorasib (100 mg/kg QD) alone, and the combination of the compound of Formula I (30 mg/kg QD) and sotorasib (100 mg/kg QD) in KRAS G12C mutant NSCLC CDX model NCI-H2122. FIG. 16B shows a graph of tumor volume versus treatment period for a KEAP1 mutant and KRAS G12C mutant NSCLC CDX model NCI-H2122 tumor xenograft model treated with vehicle (solid circles, Line 1), sotorasib alone (100 mg/kg QD, solid circles, Line 2), the compound of Formula I alone (10 mg/kg/dose BID, solid circles, Line 3), and the combination of the compound of Formula I (10 mg/kg/dose BID) and sotorasib (100 mg/kg QD, solid circles, Line 4).

### Conclusion

As illustrated by FIG. 16A and FIG. 16B, the combination of the Compound of Formula I and sotorasib demonstrated superior tumor growth inhibition relative to the respective monotherapies in the KRAS G12C mutant NSCLC CDX model NCI-H2122.

### Example 11: In Vivo Studies of the Compound of Formula I Alone, Sotorasib Alone, and the Compound of Formula I+Sotorasib Combination in a KRAS G12C Mutant CRC PDX Model CRC022

### Materials and Methods

The vehicle/control article, 100 mM acetic acid in deionized water, with pH adjustment to 4.8-5.0, was prepared and stored under ambient conditions throughout the 28-day administration in mice. The test article of the Compound of Formula I was prepared in vehicle of 100 mM acetic buffer weekly and stored under ambient conditions. The combination agent sotorasib was prepared in vehicle of 50% w/w polyethylene glycol 400 (PEG400)+50% w/w propylene glycol (PG) and stored at 2-8° C.

Female Balb/c nude mice were purchased from the Beijing Vital River Laboratory Animal Technology Co., Ltd. Mice were hosted in a special pathogen-free (SPF) environment of a vivarium facility and acclimated to their new environment for at least 3 days prior to the initiation of any experiments. Mice were between 6-8 weeks of age at the time of implantation. All procedures related to animal handling, care, and treatment in this study were performed according to the protocols and guidelines approved by the Institutional Animal Care and Use Committee (IACUC) of GenenDesign. Animal facility and program is operated under the standard of Guide for the Care and Use of Laboratory Animals (NRC, 2011) and accredited by the Association for Assessment and Accreditation of Laboratory Animal Care (AAALAC). Specifically, all portions of this study performed at GenenDesign adhered to the study protocols reviewed and approved by IACUC and applicable standard operating procedures (SOPs).

### Preparation of PDX

The CRC022 PDX model was established for pre-clinical efficacy study at GenenDesign (Shanghai, China). This PDX model was derived from a 49-year-old female Chinese CRC patient. The KRAS G12C mutation in the PDX model CRC022 was confirmed by whole exome sequencing and PCR sequencing. Tumor fragments harvested from the PDX model were implanted subcutaneously in the right flanks of female Balb/c nude mice. Mice were anesthetized with isoflurane and anesthesia was maintained throughout the implantation procedure. Mouse skin was cleaned with appropriate surgical scrub and alcohol over the right flank. A small skin incision was made using the sharp end of the trochar and a 1.5 cm subcutaneous pocket along the right lateral chest wall was formed by blunt dissection with the stylet of a 10-12 g trochar needle. Tumor fragments (15-30 mm³) were placed into the trochar needle and advanced into the subcutaneous pocket in the right flank. Trochar incision was closed with suture or a wound clip that was removed one week after closure. When tumor sizes reached a mean of approximately 200 mm³ in volume, tumor-bearing mice were randomly divided into study groups with 8 mice in each group. The randomization date was denoted as treatment day 0.

### Treatment

Treatment started on the day of randomization. The treatment start day was denoted as treatment day 0. Mice were dosed by oral administration of vehicle control solution, the Compound of Formula I monotherapy at 30 mg/kg QD, and sotorasib monotherapy at 100 mg/kg QD. One additional group received the combination treatment of the Compound of Formula I at 30 mg/kg QD and sotorasib at 100 mg/kg QD. The dosing volume was 5 mL/kg for each compound. Sotorasib was dosed one hour after the dosing of the Compound of Formula I QD in the combination group. The study was terminated at treatment day 28 as defined in the study protocol.

### Results

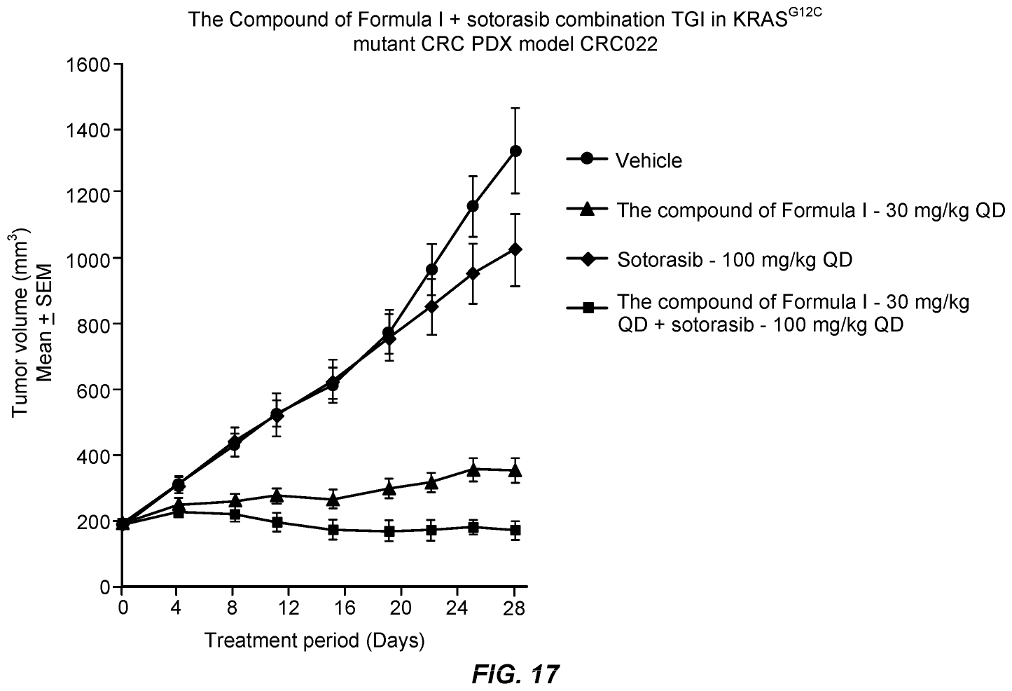

FIG. 17 shows a graph of tumor volume over a period of treatment time with a regimen of the compound of Formula I alone, sotorasib alone, and the combination of the compound of Formula I and sotorasib in KRAS G12C mutant CRC PDX model CRC022. No significant body weight change was observed in the control and treatment groups.

### CONCLUSION

As illustrated by FIG. 17, the combination of the Compound of Formula I and sotorasib demonstrated superior tumor growth inhibition relative to the respective monotherapies in the KRAS G12C mutant CRC PDX model CRC022.

Although the foregoing embodiments have been described in some detail by way of illustration and examples for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

1. A method of treating a subject having cancer comprising administering to the subject a therapeutically effective amount of a compound of Formula I or its pharmaceutically acceptable salt: 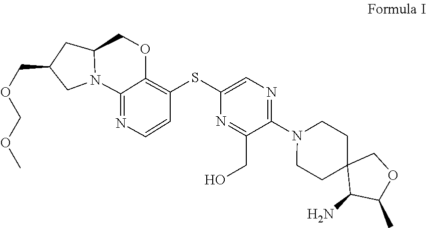 [SMILES: COCOC[C@H]1C[C@H]2COc3c(Sc4cnc(N5CCC6(CC5)CO[C@@H](C)[C@H]6N)c(CO)n4)ccnc3N2C1]
in combination with a KRAS inhibitor.

2. The method of claim 1, wherein the cancer comprises a KRAS G12, G13, H95, Q61, R68, or Y96 mutation.

3. The method of claim 1, wherein the cancer has a G12C KRAS mutation, a G12D KRAS mutation, a G12R KRAS mutation, a G12S KRAS mutation, a G12V KRAS mutation, a G12W KRAS mutation, a G13D KRAS mutation, a H95D KRAS mutation, a H95Q KRAS mutation, a H95R KRAS mutation, a Q61H KRAS mutation, a 061K KRAS mutation, or a R68S KRAS mutation.

4. The method of claim 1, wherein the cancer has a G12C KRAS mutation.

5. (canceled)

6. (canceled)

7. The method of claim 1, wherein the KRAS inhibitor is selected from the group consisting of sotorasib, adagrasib, ARS-3248, GDC-6036, BI 1701963, tipifarnib, and BBP-454.

8. (canceled)

9. (canceled)

10. (canceled)

11. (canceled)

12. The method of claim 1, wherein the method comprises administering a third MAPK pathway inhibitor.

13. The method of claim 1, wherein the administration is oral.

14. The method of claim 1, wherein the dosing of the compound of Formula I is in a range from 20 mg to 400 mg daily, 10 mg to 300 mg 10 mg to 140 mg 10 mg to 100 mg, or 5 mg to 50 mg.

15. (canceled)

16. (canceled)

17. (canceled)

18. (canceled)

19. (canceled)

20. A kit comprising a compound of Formula I or a pharmaceutically acceptable salt thereof: 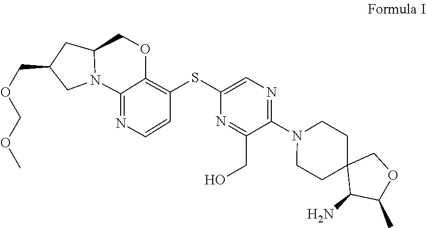 [SMILES: COCOC[C@H]1C[C@H]2COc3c(Sc4cnc(N5CCC6(CC5)CO[C@@H](C)[C@H]6N)c(CO)n4)ccnc3N2C1]
and a KRAS inhibitor.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. The method of claim 1, wherein the compound of Formula I, or a pharmaceutically acceptable salt thereof, is administered once or twice a day.

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. The method of claim 1, wherein the cancer is selected from head and neck cancer, lung cancer, stomach cancer, liver cancer, colon cancer, colorectal cancer, kidney cancer, breast cancer, pancreatic cancer, pancreatic ductal adenocarcinoma (PDAC), juvenile myelomonocytic leukemia, neuroblastoma, melanoma, and acute myeloid leukemia.
